SEQUENCE LISTING (3 sequences)

SEQ ID NO: 1 - DNA, 812 bp, Thermobifida fusca
gcgcgcaggctgctggaaaagctaatccttacgaaagaggaccgaatcctacagacgcgcttctggaggcttcaagcggacctttttctgtttctgaagaaaacgtttctagacttagcgcgtctggctttggtggcgggacaatttattacccgagagagaataacacatacggggcggtggcaatctctccggggtacacgggcacagaagcatctattgcttggcttggtgaaagaattgcttctcatggctttgttgtaatcacaattgacacaattacgacacttgatcaaccggattcaagagctgaacaattgaatgcagccctgaatcatatgatcaacagagcttcaagcacggtaagaagcagaattgatagctcaagactggcggtgatgggacatagcatgggaggcggaggcacacttagattagcctcacagagacctgatttaaaggcagcgattccgttgacgccttggcatctgaacaaaaattggtctagcgtgacagtcccgacgctcattatcggagcagatctcgatacgattgcaccggtcgcgacacatgccaaaccgttctataactcattgccgagctcaatctcaaaagcctatttagaactggatggcgccacacattttgcgccgaatattccgaacaagattatcggtaaatattcagtcgcatggttaaaaagatttgtagataatgacacgagatatacgcagttcctgtgtcctgggcctagagacggtttgttcggagaggttgaagagtatagaagcacgtgcccgttttaaaagctt

SEQ ID NO: 2 - protein, 261 aa, Thermobifida fusca
ANPYERGPNPTDALLEASSGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGLFGEVEEYRSTCPF

SEQ ID NO: 3 - protein, 264 aa, Artificial
  synthetic lipase
AGKANPYERGPNPTDALLEASSGPFSVSEENVSRLSASGFGGGTIYYPRENNTYGAVAISPGYTGTEASIAWLGERIASHGFVVITIDTITTLDQPDSRAEQLNAALNHMINRASSTVRSRIDSSRLAVMGHSMGGGGTLRLASQRPDLKAAIPLTPWHLNKNWSSVTVPTLIIGADLDTIAPVATHAKPFYNSLPSSISKAYLELDGATHFAPNIPNKIIGKYSVAWLKRFVDNDTRYTQFLCPGPRDGLFGEVEEYRSTCPF

(19) United States
(12) Patent Application Publication
(10) Pub. No.: US 2012/0258507 A1
(43) Pub. Date: Oct. 11, 2012

(54) DETERGENT COMPOSITIONS CONTAINING THERMOBIFIDA FUSCA LIPASE AND METHODS OF USE THEREOF

(75) Inventors: Christian D. Adams, Palo Alto, CA (US); Brian F. Schmidt, Half Moon Bay, CA (US)

(73) Assignee: Danisco US Inc., Palo Alto, CA (US)

(21) Appl. No.: 13/517,331

(22) PCT Filed: Dec. 14, 2010
(86) PCT No.: PCT/US2010/060253

§ 371 Date: Jun. 20, 2012

Related U.S. Application Data
(60) Provisional application No. 61/288,666, filed on Dec. 21, 2009.
(60) Provisional application No. 61/350,747, filed on Jun. 2, 2010.

(51) Int. Cl. C11D 3/386; C12P 7/62; C11D 17/00

(52) U.S. Cl. 435/135; 510/365; 510/320; 510/218; 435/264

(57) ABSTRACT

The present compositions and methods relate to a lipase cloned from Thermobifida fusca, polynucleotides encoding the lipase, and methods of use thereof. The compositions and methods have particular application in detergent cleaning compositions and methods.

### PRIORITY

The present application claims priority to U.S. Provisional Application Ser. Nos. 61/288,666, filed on Dec. 21, 2009, and 61/350,747, filed on Jun. 2, 2010, which are hereby incorporated by reference in their entirety

### TECHNICAL FIELD

The present compositions and methods relate to a lipase cloned from Thermobifida fusca, polynucleotides encoding the lipase, and methods of use, thereof.

### BACKGROUND

Current laundry detergent and/or fabric care compositions include a complex combination of active ingredients such as surfactants, enzymes (protease, amylase, lipase, and/or cellulase), bleaching agents, a builder system, suds suppressors, soil-suspending agents, soil-release agents, optical brighteners, softening agents, dispersants, dye transfer inhibition compounds, abrasives, bactericides, and perfumes.

Lipolytic enzymes, including lipases and cutinases, have been employed in detergent cleaning compositions for the removal of oily stains by hydrolyzing triglycerides to generate fatty acids. However, these enzymes are often inhibited by surfactants and other components present in cleaning composition, interfering with their ability to remove oily stains. Accordingly, the need exists for lipases and cutinases that can function in the harsh environment of cleaning compositions.

There also exists a need for more robust and efficient lipases and cutinases that are effective in performing transesterification reactions for the production of biofuels, lubricants, and other synthetic and semi-synthetic hydrocarbons. Preferably, such enzymes will utilize naturally occurring or commonly available starting materials and will not require protection and deprotection steps in a synthesis reaction, which complicate the synthesis and lead to the production of toxic waste products.

### SUMMARY

The present compositions and methods relate to lipase2 cloned from Thermobifida fusca (TfuLip2). In some embodiments, TfuLip2 has a three residue (AGK) amino terminal extension.

In one aspect of the disclosure, a recombinant TfuLip2 polypeptide is provided. In some embodiments, the recombinant TfuLip2 polypeptide is from 80% to 99% identical (e.g., 80%, 85%, 90%, 91%, 92%, 93%, 94%, 95%, 96%, 97%, 98%, or 99% identical) to the amino acid sequence of SEQ ID NO: 2. In further embodiments, the recombinant TfuLip2 polypeptide has an amino terminal extension. In some embodiments, the recombinant TfuLip2 fusion protein is at least 80% identical (e.g., 80%, 85%, 90%, 91%, 92%, 93%, 94%, 95%, 96%, 97%, 98%, 99% or 100% identical) to the amino acid sequence of SEQ ID NO: 3. In some embodiments, the TfuLip2 polypeptide is expressed in B. subtilis. The present disclosure also provides an expression vector comprising a polynucleotide encoding the TfuLip2 polypeptide in operable combination with a promoter.

In a preferred aspect of the disclosure, a detergent composition comprising a recombinant TfuLip2 polypeptide is provided. In some embodiments, the recombinant TfuLip2 polypeptide is at least 80% identical (e.g., 80%, 85%, 90%, 91%, 92%, 93%, 94%, 95%, 96%, 97%, 98%, 99%, or 100% identical) to the amino acid sequence of SEQ ID NO: 2. In further embodiments, the recombinant TfuLip2 polypeptide is at least 80% identical (e.g., 80%, 85%, 90%, 91%, 92%, 93%, 94%, 95%, 96%, 97%, 98%, 99% or 100% identical) to the amino acid sequence of SEQ ID NO:3. In some preferred embodiments, the composition comprises a surfactant (ionic or non-ionic). In some embodiments, the surfactant comprises one or more of the group consisting of sodium dodecyl benzene sulfonate, sodium hydrogenated cocoate, sodium laureth sulfate, C12-14 pareth-7, C12-15 pareth-7, sodium C12-15 pareth sulfate, C14-15 pareth-4. In some embodiments, the surfactant comprises an ionic surfactant. In some preferred embodiments, the ionic surfactant is selected from the group consisting of an anionic surfactant, a cationic surfactant, a zwitterionic surfactant, and a combination thereof. In some embodiments, the detergent is formulated at a pH of from 8.0 to 10.0. In some embodiments, the detergent is selected from the group consisting of a laundry detergent, a dishwashing detergent, and a hard-surface cleaning detergent. In some embodiments, the detergent is in a form selected from the group consisting of a liquid, a powder, a granulated solid, and a tablet. In particularly preferred embodiments, the TfuLip2 polypeptide has enzymatic activity in the detergent at a temperature from 30° C. to 40° C.

In another aspect, a detergent composition is provided, comprising: a lipase from Thermobifida fusca, and a surfactant, wherein the detergent composition is more effective in removing oily stains from a surface to be cleaned than an equivalent detergent composition lacking the lipase.

In some embodiments, the lipase is TfuLip2 lipase. In some embodiments, the lipase comprises an amino acid sequence having at least 90% amino acid sequence identity to SEQ ID NO: 2 or SEQ ID NO: 3. In some embodiments, the lipase comprises an amino acid sequence having at least 95% amino acid sequence identity to SEQ ID NO: 2 or SEQ ID NO: 3.

In some embodiments, the lipase is a recombinant lipase. In some embodiments, the lipase is a recombinant lipase expressed in Bacillus. In some embodiments, the lipase is a recombinant lipase expressed in Bacillus subtilis.

In some embodiments, the surfactant is an ionic or a non-ionic surfactant. In some embodiments, the surfactant is one or more surfactants selected from the group consisting of an anionic surfactant, a cationic surfactant, a zwitterionic surfactant, and a combination thereof. In some embodiments, the surfactant comprises one or more surfactants selected from the group consisting of sodium dodecyl benzene sulfonate, sodium hydrogenated cocoate, sodium laureth sulfate, C12-14 pareth-7, C12-15 pareth-7, sodium C12-15 pareth sulfate, and C14-15 pareth-4.

In some embodiments, the detergent composition is formulated at a pH of from about 8.0 to about 10.0. In some embodiments, the detergent composition is formulated at a pH of from about 8.2 to about 10.0.

In some embodiments, the detergent composition is selected from the group consisting of a laundry detergent, a dishwashing detergent, and a hard-surface cleaning detergent. In some embodiments, the form of the detergent composition is selected from the group consisting of a liquid, a powder, a granulated solid, and a tablet.

In some embodiments, the detergent composition is effective in hydrolyzing a lipid at a temperature of from about 30° C. to about 40° C.

In some embodiments, the detergent composition is more effective in hydrolyzing C4 to C16 substrates compared to an equivalent detergent composition comprising Pseudomonas pseudoalcaligenes lipase variant M21L (LIPOMAX™) in place of Thermobifida fusca lipase. In some embodiments, the detergent composition is more effective in hydrolyzing the C4-C16 range of substrates because it is less selective for substrates having a particular chain length.

In some embodiments, the detergent composition further comprises a protease. In some embodiments, the detergent composition further comprises a subtilisin protease. In some embodiments, the stability of the Thermobifida fusca lipase is greater than the stability of Thermomyces lanuginosus Lip3 lipase (LIPEX®) in an equivalent detergent composition comprising Thermomyces lanuginosus Lip3 lipase in place of Thermobifida fusca lipase. In some embodiments, stability of the lipase is measured in a final wash medium.

In another aspect, a method for hydrolyzing a lipid present in a soil or stain on a surface is provided, comprising contacting the surface with a detergent composition comprising a recombinant TfuLip2 polypeptide and a surfactant. The detergent compositions of the preceding paragraphs, the description, and the examples are suitable for this purpose.

In a further aspect, a method for performing a transesterification reaction is provided, comprising contacting a donor molecule with a composition comprising a recombinant TfuLip2 polypeptide. In some embodiments, the donor molecule has a C4-C16 carbon chain. In a preferred embodiment, the donor molecule has a C8 carbon chain.

These and other aspects of TfuLip2 compositions and methods will be apparent from the following description.

### DETAILED DESCRIPTION

### I. Introduction

Described are compositions and methods relating to lipase cloned from Thermobifida fusca (TfuLip2). The compositions and methods are based, in part, on the observation that cloned and expressed TfuLip2 has carboxylic ester hydrolase activity in the presence of a detergent compositions. TfuLip2 also demonstrates excellent stability in detergent compositions, even in the presence of protease. These features of TfuLip2 makes it well suited for use in a variety of cleaning applications, where the enzyme can hydrolyze lipids in the presence of surfactants and other components found in detergent compositions.

While TfuLip2 shows activity against a variety of natural and synthetic substrates, the enzyme has shown a preference for C4-C16 substrates, with peak activity against C8 substrates. This specificity makes TfuLip2 well suited for hydrolysis of short-chain triglycerides and for performing transesterification reactions involving short-chain fatty acids.

### II. Definitions

Prior to describing the present compositions and methods in detail, the following terms are defined for clarity. Terms and abbreviations not defined should be accorded their ordinary meaning as used in the art:

As used herein, a “a carboxylic ester hydrolase” (E.C. 3.1.1) refers to an enzyme that acts on carboxylic acid esters.

As used herein, a “lipase”, “lipase enzyme”, “lipolytic enzymes”, “lipolytic polypeptides”, or “lipolytic proteins” refers to an enzyme, polypeptide, or protein exhibiting a lipid degrading capability such as a capability of degrading a triglyceride or a phospholipid. The lipolytic enzyme may be, for example, a lipase, a phospholipase, an esterase or a cutinase. As used herein, lipolytic activity may be determined according to any procedure known in the art (see, e.g., Gupta et al., Biotechnol. Appl. Biochem., 37:63-71, 2003; U.S. Pat. No. 5,990,069; and International Patent Publication No. WO 96/1 8729A1).

As used herein, the term “fatty acid” refers to a carboxylic acid derived from or contained in an animal or vegetable fat or oil. Fatty acids are composed of a chain of alkyl groups typically containing from 4-22 carbon atoms and characterized by a terminal carboxyl group (—COOH). Fatty acids may be saturated or unsaturated, and solid, semisolid, or liquid.

As used herein, the term “triglyceride” refers to any naturally occurring ester of a fatty acid and glycerol. Triglycerides are the chief constituents of fats and oils. The have the general formula of CH₂(OOCR₁)CH(OOCR₂)CH₂(OOCR₃), where R₁, R₂, and R₃ may be of different chain length.

As used herein, “acyl” is the general name for an organic acid group (RCO—), generally obtained by removing the —OH group from a carboxylic acid.

As used herein, the term “acylation” refers to a chemical transformation which substitutes/adds an acyl group into a molecule, generally at the side of an —OH group.

As used herein, an “acyl chain substrate” is a donor molecule for a carboxylic ester hydrolase (e.g., cutinase, lipase, acyltransferase, transesterase, and the like). The substrate may be described in terms of its carbon-chain length. For example, a C4 substrate/donor has a chain-length of 4 carbons, a C8 substrate/donor has a chain-length of 8 carbons, and the like.

As used herein, the term “transferase” refers to an enzyme that catalyzes the transfer of a molecule or group (e.g., an acyl group) to a substrate.

As used herein, “leaving group” refers to the nucleophile which is cleaved from the acyl donor upon substitution by another nucleophile.

As used herein, the phrase “detergent stability” refers to the stability of a specified detergent composition component (such as a hydrolytic enzyme) in a detergent composition mixture. Exemplary hydrolytic enzymes are proteases, and stability can refer to the resistance of a lipase to hydrolysis by a protease. The stability of the present lipase may be compared to the stability of a standard, for example, a commercially available lipase such as LIPOMAX™ or LIPEX™, which are described, herein.

As used herein, a “perhydrolase” is an enzyme capable of catalyzing a reaction that results in the formation of a peracid suitable for applications such as cleaning, bleaching, and disinfecting.

As used herein, the term “aqueous,” as used in the phrases “aqueous composition” and “aqueous environment,” refers to a composition that is made up of at least 50% water. An aqueous composition may contain at least 50% water, at least 60% water, at least 70% water, at least 80% water, at least 90% water, at least 95% water, at least 97% water, at least 99% water, or even at least 99% water.

As used herein, the term “surfactant” refers to any compound generally recognized in the art as having surface active qualities. Surfactants generally include anionic, cationic, nonionic, and zwitterionic compounds, which are further described, herein.

As used herein, “surface property” is used in reference to electrostatic charge, as well as properties such as the hydrophobicity and hydrophilicity exhibited by the surface of a protein.

The term “oxidation stability” refers to lipases of the present disclosure that retain a specified amount of enzymatic activity over a given period of time under conditions prevailing during the lipolytic, hydrolyzing, cleaning or other process disclosed herein, for example while exposed to or contacted with bleaching agents or oxidizing agents. In some embodiments, the lipases retain at least about 50%, about 60%, about 70%, about 75%, about 80%, about 85%, about 90%, about 92%, about 95%, about 96%, about 97%, about 98%, or about 99% lipolytic activity after contact with a bleaching or oxidizing agent over a given time period, for example, at least about 1 minute, about 3 minutes, about 5 minutes, about 8 minutes, about 12 minutes, about 16 minutes, about 20 minutes, etc.

The term “chelator stability” refers to lipases of the present disclosure that retain a specified amount of enzymatic activity over a given period of time under conditions prevailing during the lipolytic, hydrolyzing, cleaning or other process disclosed herein, for example while exposed to or contacted with chelating agents. In some embodiments, the lipases retain at least about 50%, about 60%, about 70%, about 75%, about 80%, about 85%, about 90%, about 92%, about 95%, about 96%, about 97%, about 98%, or about 99% lipolytic activity after contact with a chelating agent over a given time period, for example, at least about 10 minutes, about 20 minutes, about 40 minutes, about 60 minutes, about 100 minutes, etc.

The terms “thermal stability” and “thermostable” refer to lipases of the present disclosure that retain a specified amount of enzymatic activity after exposure to identified temperatures over a given period of time under conditions prevailing during the lipolytic, hydrolyzing, cleaning or other process disclosed herein, for example while exposed altered temperatures. Altered temperatures include increased or decreased temperatures. In some embodiments, the lipases retain at least about 50%, about 60%, about 70%, about 75%, about 80%, about 85%, about 90%, about 92%, about 95%, about 96%, about 97%, about 98%, or about 99% lipolytic activity after exposure to altered temperatures over a given time period, for example, at least about 60 minutes, about 120 minutes, about 180 minutes, about 240 minutes, about 300 minutes, etc.

The term “cleaning activity” refers to the cleaning performance achieved by the lipase under conditions prevailing during the lipolytic, hydrolyzing, cleaning or other process disclosed herein. In some embodiments, cleaning performance is determined by the application of various cleaning assays concerning enzyme sensitive stains, for example grass, blood, milk, or egg protein as determined by various chromatographic, spectrophotometric or other quantitative methodologies after subjection of the stains to standard wash conditions. Exemplary assays include, but are not limited to those described in WO 99/34011, and U.S. Pat. No. 6,605,458 (both of which are herein incorporated by reference), as well as those methods included in the Examples.

The term “cleaning effective amount” of a lipase refers to the quantity of lipase described hereinbefore that achieves a desired level of enzymatic activity in a specific cleaning composition. Such effective amounts are readily ascertained by one of ordinary skill in the art and are based on many factors, such as the particular lipase used, the cleaning application, the specific composition of the cleaning composition, and whether a liquid or dry (e.g., granular, bar) composition is required, etc.

The term “cleaning adjunct materials,” as used herein, means any liquid, solid or gaseous material selected for the particular type of cleaning composition desired and the form of the product (e.g., liquid, granule, powder, bar, paste, spray, tablet, gel; or foam composition), which materials are also preferably compatible with the lipase enzyme used in the composition. In some embodiments, granular compositions are in “compact” form, while in other embodiments, the liquid compositions are in a “concentrated” form.

As used herein, “cleaning compositions” and “cleaning formulations” refer to admixtures of chemical ingredients that find use in the removal of undesired compounds (e.g., soil or stains) from items to be cleaned, such as fabric, dishes, contact lenses, other solid surfaces, hair, skin, teeth, and the like. The composition or formulations may be in the form of a liquid, gel, granule, powder, or spray, depending on the surface, item or fabric to be cleaned, and the desired form of the composition or formulation.

As used herein, the terms “detergent composition” and “detergent formulation” refer to mixtures of chemical ingredients intended for use in a wash medium for the cleaning of soiled objects. Detergent compositions/formulations generally include at least one surfactant, and may optionally include hydrolytic enzymes, oxido-reductases, builders, bleaching agents, bleach activators, bluing agents and fluorescent dyes, caking inhibitors, masking agents, enzyme activators, antioxidants, and solubilizers.

As used herein, “dishwashing composition” refers to all forms of compositions for cleaning dishware, including cutlery, including but not limited to granular and liquid forms. In some embodiments, the dishwashing composition is an “automatic dishwashing” composition that finds use in automatic dish washing machines. It is not intended that the present disclosure be limited to any particular type or dishware composition. Indeed, the present disclosure finds use in cleaning dishware (e.g., dishes, including, but not limited to plates, cups, glasses, bowls, etc.) and cutlery (e.g., utensils, including but not limited to spoons, knives, forks, serving utensils, etc.) of any material, including but not limited to ceramics, plastics, metals, china, glass, acrylics, etc. The term “dishware” is used herein in reference to both dishes and cutlery.

As used herein, the term “bleaching” refers to the treatment of a material (e.g., fabric, laundry, pulp, etc.) or surface for a sufficient length of time and under appropriate pH and temperature conditions to effect a brightening (i.e., whitening) and/or cleaning of the material. Examples of chemicals suitable for bleaching include but are not limited to ClO₂, H₂O₂, peracids, NO₂, etc.

As used herein, “wash performance” of a variant lipase refers to the contribution of a variant lipase to washing that provides additional cleaning performance to the detergent without the addition of the variant lipase to the composition. Wash performance is compared under relevant washing conditions.

The term “relevant washing conditions” is used herein to indicate the conditions, particularly washing temperature, time, washing mechanics, sud concentration, type of detergent and water hardness, actually used in households in a dish or laundry detergent market segment.

As used herein, the term “disinfecting” refers to the inhibition or killing of microbes on the surfaces of items. It is not intended that the present disclosure be limited to any particular surface, item, or contaminant(s) or microbes to be removed.

The “compact” form of the cleaning compositions herein is best reflected by density and, in terms of composition, by the amount of inorganic filler salt. Inorganic filler salts are conventional ingredients of detergent compositions in powder form. In conventional detergent compositions, the filler salts are present in substantial amounts, typically about 17 to about 35% by weight of the total composition. In contrast, in compact compositions, the filler salt is present in amounts not exceeding about 15% of the total composition. In some embodiments, the filler salt is present in amounts that do not exceed about 10%, or more preferably, about 5%, by weight of the composition. In some embodiments, the inorganic filler salts are selected from the alkali and alkaline-earth-metal salts of sulfates and chlorides. In some embodiments, a preferred filler salt is sodium sulfate.

As used herein, the terms “textile” or “textile material” refer to woven fabrics, as well as staple fibers and filaments suitable for conversion to or use as yarns, woven, knit, and non-woven fabrics. The term encompasses yarns made from natural, as well as synthetic (e.g., manufactured) fibers.

As used herein, the terms “purified” and “isolated” refer to the physical separation of a subject molecule, such as TfuLip2, from other molecules, such as proteins, nucleic acids, lipids, media components, and the like. Once purified or isolated, a subject molecule may represent at least 50%, and even at least 60%, at least 70%, at least 80%, at least 85%, at least 90%, at least 95%, or more, of the total amount of material in a sample (wt/wt).

As used herein, a “polypeptide” refers to a molecule comprising a plurality of contiguous amino acid residues linked through peptide bonds. The terms “polypeptide,” “peptide,” and “protein” are used interchangeably. Proteins may be optionally be modified (e.g., glycosylated, phosphorylated, acylated, farnesylated, prenylated, sulfonated, and the like) to add functionality. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The terms “polynucleotide” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence. Unless otherwise indicated, nucleic acid sequences are presented in a 5′-to-3′ orientation.

As used herein, the terms “wild-type” and “native” refer to polypeptides or polynucleotides that are found in nature.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

As used herein, a “variant polypeptide” refers to a polypeptide that is derived from a parent (or reference) polypeptide by the substitution, addition, or deletion, of one or more amino acids, typically by recombinant DNA techniques. Variant polypeptides may differ from a parent polypeptide by a small number of amino acid residues and may be defined by their level of primary amino acid sequence homology/identity with a parent polypeptide. Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent polypeptide.

Sequence identity may be determined using known programs such as BLAST, ALIGN, and CLUSTAL using standard parameters. (See, e.g., Altschul et al. (1990) J. Mol. Biol. 215:403-410; Henikoff et al. (1989) Proc. Natl. Acad. Sci. USA 89:10915; Karin et al. (1993) Proc. Natl. Acad. Sci. USA 90:5873; and Higgins et al. (1988) Gene 73:237-244). Software for performing BLAST analyses is publicly available through the National Center for Biotechnology Information. Also, databases may be searched using FASTA (Pearson et al. (1988) Proc. Natl. Acad. Sci. USA 85:2444-2448). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution.

As used herein, a “variant polynucleotide” encodes a variant polypeptide, has a specified degree of homology/identity with a parent polynucleotide, or hybridized under stringent conditions to a parent polynucleotide or the complement, thereof. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent polynucleotide. Methods for determining percent identity are known in the art and described immediately above.

The term “derived from” encompasses the terms “originated from,” “obtained from,” “obtainable from,” “isolated from,” and “created from,” and generally indicates that one specified material find its origin in another specified material or has features that can be described with reference to the another specified material.

As used herein, the term “hybridization” refers to the process by which a strand of nucleic acid joins with a complementary strand through base pairing, as known in the art

As used herein, the phrase “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Alternatively, or in addition, hybridization conditions can be based upon the salt or ionic strength conditions of hybridization and/or one or more stringency washes, e.g.: 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g., relatively low salt and/or high temperature conditions are used). As used herein, stringent conditions are defined as 50° C. and 0.2×SSC (1×SSC=0.15 M NaCl, 0.015 M sodium citrate, pH 7.0).

The phrases “substantially similar” and “substantially identical” in the context of at least two nucleic acids or polypeptides means that a polynucleotide or polypeptide comprises a sequence that has at least about 90%, at least about 91%, at least about 92%, at least about 93%, at least about 94%, at least about 95%, at least about 96%, at least about 97%, at least about 98%, or even at least about 99% identical to a parent or reference sequence, or does not include amino acid substitutions, insertions, deletions, or modifications made only to circumvent the present description without adding functionality.

As used herein, an “expression vector” refers to a DNA construct containing a DNA sequence that encodes a specified polypeptide and is operably linked to a suitable control sequence capable of effecting the expression of the polypeptides in a suitable host. Such control sequences include a promoter to effect transcription, an optional operator sequence to control such transcription, a sequence encoding suitable mRNA ribosome binding sites and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself.

The term “recombinant,” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally recombinant nucleic acids, polypeptides, and cells based thereon, have been manipulated by man such that they are not identical to related nucleic acids, polypeptides, and cells found in nature.

A “signal sequence” refers to a sequence of amino acids bound to the N-terminal portion of a polypeptide, and which facilitates the secretion of the mature form of the protein from the cell. The mature form of the extracellular protein lacks the signal sequence which is cleaved off during the secretion process.

The term “selective marker” or “selectable marker” refers to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell.

The term “regulatory element” as used herein refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

As used herein, “host cells” are generally prokaryotic or eukaryotic hosts which are transformed or transfected with vectors constructed using recombinant DNA techniques known in the art. Transformed host cells are capable of either replicating vectors encoding the protein variants or expressing the desired protein variant. In the case of vectors which encode the pre- or prepro-form of the protein variant, such variants, when expressed, are typically secreted from the host cell into the host cell medium.

The term “introduced” in the context of inserting a nucleic acid sequence into a cell, means transformation, transduction or transfection. Means of transformation include protoplast transformation, calcium chloride precipitation, electroporation, naked DNA and the like as known in the art. (See, Chang and Cohen (1979) Mol. Gen. Genet., 168:111-115; Smith et al. (1986) Appl. Env. Microbiol., 51:634; and the review article by Ferrari et al., in Harwood, Bacillus, Plenum Publishing Corporation, pp. 57-72, 1989).

The terms “selectable marker” or “selectable gene product” as used herein refer to the use of a gene which encodes an enzymatic activity that confers resistance to an antibiotic or drug upon the cell in which the selectable marker is expressed.

Other technical and scientific terms have the same meaning as commonly understood by one of ordinary skill in the art to which this disclosure pertains (see, e.g., Singleton and Sainsbury, Dictionary of Microbiology and Molecular Biology, 2d Ed., John Wiley and Sons, NY (1994); and Hale and Marham, The Harper Collins Dictionary of Biology, Harper Perennial, N.Y. (1991).

The singular terms “a,” “an,” and “the” include the plural reference unless the context clearly indicates otherwise.

Headings are provided for convenience and should not be construed as limitations. The description included under one heading may apply to the specification as a whole.

### III. TfuLip2 Polypeptides and Polynucleotides

A. TfuLip2 Polypeptides

In one aspect, the present compositions and methods provide a recombinant TfuLip2 polypeptide or a variant thereof. An exemplary TfuLip2 polypeptide was isolated from Thermobifida fusca (GENBANK Accession No. YP_{—}288944). The mature TfuLip2 polypeptide has the amino acid sequence of SEQ ID NO: 3. Similar, substantially identical TfuLip2 polypeptides may occur in nature, e.g., in other strains or isolates of T. fusca. These and other recombinant TfuLip2 polypeptides are encompassed by the present compositions and methods.

In some embodiments, the recombinant TfuLip2 polypeptide is a variant TfuLip2 polypeptide having a specified degree of amino acid sequence homology to the exemplified TfuLip2 polypeptide, e.g., at least 70%, at least 75%, at least 80%, at least 85%, at least 90%, at least 91%, at least 92%, at least 93%, at least 94%, at least 95%, at least 96%, at least 97%, at least 98%, or even at least 99% sequence homology to the amino acid sequence of SEQ ID NO: 2 (infra) or SEQ ID NO: 3. Homology can be determined by amino acid sequence alignment, e.g., using a program such as BLAST, ALIGN, or CLUSTAL, as described herein.

In some embodiments, the recombinant TfuLip2 polypeptide includes substitutions that do not substantially affect the structure and/or function of the polypeptide. Exemplary substitutions are conservative mutations, as summarized in Table I.

TABLE I
Amino Acid Substitutions

| Original | | |
|---|---|---|
| Residue | Code | Acceptable Substitutions |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, beta-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Substitutions involving naturally occurring amino acids are generally made by mutating a nucleic acid encoding a recombinant TfuLip2 polypeptide, and then expressing the variant polypeptide in an organism. Substitutions involving non-naturally occurring amino acids or chemical modifications to amino acids are generally made by chemically modifying a recombinant TfuLip2 polypeptide after it has been synthesized by an organism.

In some embodiments, variant recombinant TfuLip2 polypeptides are substantially identical to SEQ ID NO: 3, meaning that they do not include amino acid substitutions, insertions, or deletions that do not significantly affect the structure, function or expression of the polypeptide. Such variant recombinant TfuLip2 polypeptides include those designed only to circumvent the present description.

In some embodiments, the recombinant TfuLip2 polypeptide (including a variant, thereof) has carboxylic ester hydrolase activity, which includes lipase, esterase, transesterase, and/or acyltransferase activity. Carboxylic ester hydrolase activity can be determined and measured using the assays described herein, or by other assays known in the art. In some embodiments, the recombinant TfuLip2 polypeptide has activity in the presence of a detergent composition.

TfuLip2 polypeptides include fragments of “full-length” TfuLip2 polypeptides that retain carboxylic ester hydrolase activity. Such fragments preferably retain the active site of the full-length polypeptides but may have deletions of non-critical amino acid residues. The activity of fragments can readily be determined using the assays described, herein, or by other assays known in the art. In some embodiments, the fragments of TfuLip2 polypeptides retain carboxylic ester hydrolase activity in the presence of a detergent composition.

In some embodiments, the TfuLip2 polypeptide is fused to a signal peptide for directing the extracellular secretion of the TfuLip2 polypeptide. In some embodiments, the TfuLip2 polypeptide is expressed in a heterologous organism, i.e., an organism other than Bacillus subtilis. Exemplary heterologous organisms are Gram(+) bacteria such as Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces lividans, or Streptomyces murinus; Gram(−) bacteria such as E. coli; yeast such as Saccharomyces spp. or Schizosaccharomyces spp., e.g. Saccharomyces cerevisiae; and filamentous fungi such as Aspergillus spp., e.g., Aspergillus oryzae or Aspergillus niger, and Trichoderma reesei. Methods from transforming nucleic acids into these organisms are well known in the art. A suitable procedure for transformation of Aspergillus host cells is described in EP 238 023.

In particular embodiments, the TfuLip2 polypeptide is expressed in a heterologous organism as a secreted polypeptide, in which case, the compositions and method encompass a method for expressing a TfuLip2 polypeptide as a secreted polypeptide in a heterologous organism.

B. TfuLip2 Polynucleotides

Another aspect of the compositions and methods is a polynucleotide that encodes a TfuLip2 polypeptide (including variants and fragments, thereof), provided in the context of an expression vector for directing the expression of a TfuLip2 polypeptide in a heterologous organism, such as those identified, herein. The polynucleotide that encodes a TfuLip2 polypeptide may be operably-linked to regulatory elements (e.g., a promoter, terminator, enhancer, and the like) to assist in expressing the encoded polypeptides.

An exemplary polynucleotide sequence encoding a TfuLip2 polypeptide has the nucleotide sequence of SEQ ID NO: 1. Similar, including substantially identical, polynucleotides encoding TfuLip2 polypeptides and variants may occur in nature, e.g., in other strains or isolates of T. fusca. In view of the degeneracy of the genetic code, it will be appreciated that polynucleotides having different nucleotide sequences may encode the same TfuLip2 polypeptides, variants, or fragments.

In some embodiments, polynucleotides encoding TfuLip2 polypeptides have a specified degree of amino acid sequence homology to the exemplified polynucleotide encoding a TfuLip2 polypeptide, e.g., at least 70%, at least 75%, at least 80%, at least 85%, at least 90%, at least 91%, at least 92%, at least 93%, at least 94%, at least 95%, at least 96%, at least 97%, at least 98%, or even at least 99% sequence homology to the amino acid sequence of SEQ ID NO: 1. Homology can be determined by amino acid sequence alignment, e.g., using a program such as BLAST, ALIGN, or CLUSTAL, as described herein.

In some embodiments, the polynucleotide that encodes a TfuLip2 polypeptide is fused in frame behind (i.e., downstream of) a coding sequence for a signal peptide for directing the extracellular secretion of a TfuLip2 polypeptide. Heterologous signal sequences include those from bacterial cellulase genes. Expression vectors may be provided in a heterologous host cell suitable for expressing a TfuLip2 polypeptide, or suitable for propagating the expression vector prior to introducing it into a suitable host cell.

In some embodiments, polynucleotides encoding TfuLip2 polypeptides hybridize to the exemplary polynucleotide of SEQ ID NO: 1 (or the complement, thereof) under specified hybridization conditions. Exemplary conditions are stringent condition and highly stringent conditions, which are described, herein.

TfuLip2 polynucleotides may be naturally occurring or synthetic (i.e., man-made), and may be codon-optimized for expression in a different host, mutated to introduce cloning sites, or otherwise altered to add functionality.

### IV. Activities and Properties of TfuLip2 Polypeptides

The TfuLip2 polypeptides disclosed herein may have enzymatic activity over a broad range of pH conditions. In certain embodiments the disclosed TfuLip2 polypeptides have enzymatic activity from about pH 4 to about pH 11.5. In preferred embodiments, TfuLip2 is active from about pH 8 to about pH 10. It should be noted that the pH values described herein may vary by ±0.2. For example a pH value of about 8 could vary from pH 7.8 to pH 8.2.

The TfuLip2 polypeptides disclosed herein may have enzymatic activity over a wide range of temperatures, e.g., from 10° C. or lower to about 50° C. In certain embodiments, the optimum temperature range for TfuLip2 lipase is from about 10° C. to about 20° C., from about 20° C. to about 30° C., from about 30° C. to about 40° C., or from about 40° C. to about 50° C. It should be noted that the temperature values described herein may vary by ±0.2° C. For example a temperature of about 10° C. could vary from 9.8° C. to 10.2° C.

As shown in Example 3, the activity of TfuLip2 polypeptide was highest using a C8 substrate, but activity was observed using C4 and C16 substrates. In contrast, the commercially produced lipase LIPOMAX™ (i.e., Pseudomonas pseudoalcaligenes lipase variant M21L, Danisco US. Inc, Genencor Division, Palo Alto, Calif., USA) had a preference for C10 substrates, with activity falling off rapidly with smaller (e.g., C8) or larger (e.g., C16) substrates (not shown). Therefore, TfuLip2 polypeptide appears to be less selective that LIPOMAX™ for substrates of a particular length, while having a preference for substrates with a shorter chain length than LIPOMAX™. In fact, TfuLip2 showed hydrolysis activity against an exemplary oily stain material, in the presence of detergent compositions both in solution (Example 4) and when the stain was present on fabric (Example 5).

In addition to having excellent cleaning performance and broad substrate specificity, TfuLip2 lipase is also stable in detergent compositions, particularly in the presence of protease. The stability of TfuLip2 lipase can conveniently be measured against the stability of LIPEX™ using equivalent assay conditions. Exemplary assay conditions are described, herein (including but not limited to Example 14). Stability may be assayed under final wash conditions or in a concentrated storage form of a detergent formulation.

In some embodiments, TfuLip2 lipase is at least about 10%, at least about 15%, or even at least about 20% more stable than LIPEX™, over a period of about a week in an equivalent detergent composition lacking protease. In some embodiments, TfuLip2 lipase is at least about 10%, at least about 15%, or even at least about 20% more stable than LIPEX™, over a period of about fifteen days in an equivalent detergent composition lacking protease. Exemplary detergent compositions are OMO™ Small and Mighty and ARIEL™. In some embodiments, TfuLip2 lipase is at least about 1.2-fold, at least about 1.3-fold, at least about 1.4-fold, or even at least about 1.5-fold more stable than LIPEX™, over a period of about a week in an equivalent detergent composition lacking protease. In some embodiments, TfuLip2 lipase is at least about 1.2-fold, at least about 1.3-fold, at least about 1.4-fold, or even at least about 1.5-fold more stable than LIPEX™, over a period of about fifteen days in an equivalent detergent composition lacking protease. Exemplary detergent compositions are OMO™ Small and Mighty and ARIEL™.

In some embodiments, TfuLip2 lipase is at least about 100%, at least about 150%, at least about 200%, at least about 250%, at least about 300%, at least about 350%, at least about 400%, at least about 450%, or even at least about 500% more stable than LIPEX™, over a period of about a week in an equivalent detergent composition including protease. In some embodiments, TfuLip2 lipase is at least about 100%, at least about 150%, at least about 200%, at least about 250%, at least about 300%, at least about 350%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1,000%, at least about 1,100%, at least about 1,200%, at least about 1,300%, at least about 1,400%, at least about 1,500%, at least about 1,600%, at least about 1,700%, or even at least about 1,800% more stable than LIPEX™, over a period of about fifteen days in an equivalent detergent composition including protease. Exemplary detergent compositions are OMO™ Small and Mighty and ARIEL™. In some embodiments, TfuLip2 lipase is at least about 2-fold, at least about 2.5-fold, at least about 3-fold, at least about 3.5-fold, at least about 4-fold, at least about 4.5-fold, or even at least about 5-fold more stable than LIPEX™, over a period of about a week in an equivalent detergent composition including protease. In some embodiments, TfuLip2 lipase is at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, or even at least about 18-fold more stable than LIPEX™, over a period of about fifteen days in an equivalent detergent composition including protease. Exemplary detergent compositions are OMO™ Small and Mighty and ARIEL™.

These and other properties and advantages of TfuLip2 lipase are described, herein.

### V. Detergent Compositions Comprising a TfuLip2 Polypeptide

An aspect of the compositions and methods disclosed herein is a detergent composition comprising a TfuLip2 polypeptide (including variants or fragments, thereof) and methods for using such compositions in cleaning applications. Cleaning applications include, but are not limited to, laundry or textile cleaning, dishwashing (manual and automatic), stain pre-treatment, and the like. Particular applications are those where lipids are a component of the soils or stains to be removed. Detergent compositions typically include an effective amount of TfuLip2 or a variant thereof, e.g., at least 0.0001 weight-percent, from about 0.0001 to about 1, from about 0.001 to about 0.5, from about 0.01 to about 0.1 weight-percent, or even from about 0.1 to about 1 weight-percent, or more. Detergent compositions having a concentration from about 0.4 g/L to about 2.2 g/L, from about 0.4 g/L to about 2.0 g/L, from about 0.4 g/L to about 1.7 g/L, from about 0.4 g/L to about 1.5 g/L, from about 0.4 g/L to about 1 g/L, from about 0.4 g/L to about 0.8 g/L, or from about 0.4 g/L to about 0.5 g/L may be mixed with an effective amount of a TfuLip2 lipase. The detergent composition may also be present at a concentration of about 0.4 ml/L to about 2.6 ml/L, from about 0.4 ml/L to about 2.0 ml/L, from about 0.4 ml/L to about 1.5 m/L, from about 0.4 ml/L to about 1 ml/L, from about 0.4 ml/L to about 0.8 ml/L, or from about 0.4 ml/L to about 0.5 ml/L.

Unless otherwise noted, all component or composition levels provided herein are made in reference to the active level of that component or composition, and are exclusive of impurities, for example, residual solvents or by-products, which may be present in commercially available sources. Enzyme components weights are based on total active protein. All percentages and ratios are calculated by weight unless otherwise indicated. All percentages and ratios are calculated based on the total composition unless otherwise indicated. In the exemplified detergent compositions, the enzymes levels are expressed by pure enzyme by weight of the total composition and unless otherwise specified, the detergent ingredients are expressed by weight of the total compositions.

In some embodiments, the detergent composition comprises one or more surfactants, which may be non-ionic, semi-polar, anionic, cationic, zwitterionic, or combinations and mixtures thereof. The surfactants are typically present at a level of from about 0.1% to 60% by weight. Exemplary surfactants include but are not limited to sodium dodecylbenzene sulfonate, C12-14 pareth-7, C12-15 pareth-7, sodium C12-15 pareth sulfate, C14-15 pareth-4, sodium laureth sulfate (e.g., Steol CS-370), sodium hydrogenated cocoate, C12 ethoxylates (Alfonic 1012-6, Hetoxol LA7, Hetoxol LA4), sodium alkyl benzene sulfonates (e.g., Nacconol 90G), and combinations and mixtures thereof.

Anionic surfactants that may be used with the detergent compositions described herein include but are not limited to linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. Detergent compositions may also contain 0-40% of nonionic surfactant such as alcohol ethoxylate (AEO or AE), carboxylated alcohol ethoxylates, nonylphenol ethoxylate, alkylpolyglycoside, alkyldimethylamine oxide, ethoxylated fatty acid monoethanolamide, fatty acid monoethanolamide, polyhydroxy alkyl fatty acid amide (e.g., as described in WO 92/06154), and combinations and mixtures thereof.

Nonionic surfactants that may be used with the detergent compositions described herein include but are not limited to polyoxyethylene esters of fatty acids, polyoxyethylene sorbitan esters (e.g., TWEENs), polyoxyethylene alcohols, polyoxyethylene isoalcohols, polyoxyethylene ethers (e.g., TRITONs and BRIJ), polyoxyethylene esters, polyoxyethylene-p-tert-octylphenols or octylphenyl-ethylene oxide condensates (e.g., NONIDET P40), ethylene oxide condensates with fatty alcohols (e.g., LUBROL), polyoxyethylene nonylphenols, polyalkylene glycols (SYNPERONIC F108), sugar-based surfactants (e.g., glycopyranosides, thioglycopyranosides), and combinations and mixtures thereof.

The detergent compositions disclosed herein may have mixtures that include but are not limited to 5-15% anionic surfactants, <5% nonionic surfactants, cationic surfactants, phosphonates, soap, enzymes, perfume, butylphenyl methylptopionate, geraniol, zeolite, polycarboxylates, hexyl cinnamal, limonene, cationic surfactants, citronellol, and benzisothiazolinone.

Detergent compositions may additionally include one or more detergent builders or builder systems, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, a tarnish inhibitor, an optical brightener, a fabric conditioner, and a perfume. The detergent compositions may also include enzymes, including but not limited to proteases, amylases, cellulases, lipases, or additional carboxylic ester hydrolases. The pH of the detergent compositions should be neutral to basic, as described, herein.

In some embodiments incorporating at least one builder, the detergent compositions comprise at least about 1%, from about 3% to about 60%, or even from about 5% to about 40% builder, by weight (i.e., wt/wt, weight-percent) of the cleaning composition. Builders may include, but are not limited to, the alkali metal, ammonium and alkanolammonium salts of polyphosphates, alkali metal silicates, alkaline earth and alkali metal carbonates, aluminosilicates, polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof. Indeed, it is contemplated that any suitable builder will find use in various embodiments of the present disclosure.

In some embodiments, the builders form water-soluble hardness ion complexes (e.g., sequestering builders), such as citrates and polyphosphates (e.g., sodium tripolyphosphate and sodium tripolyphospate hexahydrate, potassium tripolyphosphate, and mixed sodium and potassium tripolyphosphate, etc.). It is contemplated that any suitable builder will find use in the present disclosure, including those known in the art (see e.g., EP 2 100 949).

As indicated herein, in some embodiments, the cleaning compositions described herein further comprise adjunct materials including, but not limited to, surfactants, builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (see e.g., U.S. Pat. Nos. 6,610,642, 6,605,458, 5,705,464, 5,710,115, 5,698,504, 5,695,679, 5,686,014 and 5,646,101, all of which are incorporated herein by reference). Embodiments of specific cleaning composition materials are exemplified in detail below. In embodiments in which the cleaning adjunct materials are not compatible with the TfuLip2 variants in the cleaning compositions, then suitable methods of keeping the cleaning adjunct materials and the lipase(s) separated (i.e., not in contact with each other) until combination of the two components is appropriate are used. Such separation methods include any suitable method known in the art (e.g., gelcaps, encapsulation, tablets, physical separation, etc.).

The cleaning compositions described herein are advantageously employed for example, in laundry applications, hard surface cleaning, dishwashing applications, as well as cosmetic applications such as dentures, teeth, hair and skin. In addition, due to the unique advantages of increased effectiveness in lower temperature solutions, the TfuLip2 enzymes described herein are ideally suited for laundry applications. Furthermore, the TfuLip2 enzymes may find use in granular and liquid compositions.

The TfuLip2 polypeptides described herein may also find use cleaning in additive products. In some embodiments, low temperature solution cleaning applications find use. In some embodiments, the present disclosure provides cleaning additive products including at least one disclosed TfuLip2 polypeptide is ideally suited for inclusion in a wash process when additional bleaching effectiveness is desired. Such instances include, but are not limited to low temperature solution cleaning applications. In some embodiments, the additive product is in its simplest form, one or more lipases. In some embodiments, the additive is packaged in dosage form for addition to a cleaning process. In some embodiments, the additive is packaged in dosage form for addition to a cleaning process where a source of peroxygen is employed and increased bleaching effectiveness is desired. Any suitable single dosage unit form finds use with the present disclosure, including but not limited to pills, tablets, gelcaps, or other single dosage units such as pre-measured powders or liquids. In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials. Acidic fillers find use to reduce pH. Alternatively, in some embodiments, the cleaning additive includes adjunct ingredients, as more fully described below.

The present cleaning compositions and cleaning additives require an effective amount of at least one of the TfuLip2 polypeptides described herein, alone or in combination with other lipases and/or additional enzymes. The required level of enzyme is achieved by the addition of one or more disclosed TfuLip2 polypeptide. Typically the present cleaning compositions will comprise at least about 0.0001 weight percent, from about 0.0001 to about 10, from about 0.001 to about 1, or even from about 0.01 to about 0.1 weight percent of at least one of the disclosed TfuLip2 polypeptides.

The cleaning compositions herein are typically formulated such that, during use in aqueous cleaning operations, the wash water will have a pH of from about 5.0 to about 11.5 or even from about 7.5 to about 10.5. Liquid product formulations are typically formulated to have a neat pH from about 3.0 to about 9.0 or even from about 3 to about 5. Granular laundry products are typically formulated to have a pH from about 9 to about 11. Techniques for controlling pH at recommended usage levels include the use of buffers, alkalis, acids, etc., and are well known to those skilled in the art.

Suitable low pH cleaning compositions typically have a neat pH of from about 3 to about 5, and are typically free of surfactants that hydrolyze in such a pH environment. Such surfactants include sodium alkyl sulfate surfactants that comprise at least one ethylene oxide moiety or even from about 1 to about 16 moles of ethylene oxide. Such cleaning compositions typically comprise a sufficient amount of a pH modifier, such as sodium hydroxide, monoethanolamine or hydrochloric acid, to provide such cleaning composition with a neat pH of from about 3 to about 5. Such compositions typically comprise at least one acid stable enzyme. In some embodiments, the compositions are liquids, while in other embodiments, they are solids. The pH of such liquid compositions is typically measured as a neat pH. The pH of such solid compositions is measured as a 10% solids solution of said composition wherein the solvent is distilled water. In these embodiments, all pH measurements are taken at 20° C., unless otherwise indicated.

In some embodiments, when the TfuLip2 polypeptide is employed in a granular composition or liquid, it is desirable for the TfuLip2 polypeptide to be in the form of an encapsulated particle to protect the TfuLip2 polypeptide from other components of the granular composition during storage. In addition, encapsulation is also a means of controlling the availability of the TfuLip2 polypeptide during the cleaning process. In some embodiments, encapsulation enhances the performance of the TfuLip2 polypeptide and/or additional enzymes. In this regard, the TfuLip2 polypeptide of the present disclosure are encapsulated with any suitable encapsulating material known in the art. In some embodiments, the encapsulating material typically encapsulates at least part of the catalyst for the TfuLip2 polypeptides described herein. Typically, the encapsulating material is water-soluble and/or water-dispersible. In some embodiments, the encapsulating material has a glass transition temperature (Tg) of 0° C. or higher. Glass transition temperature is described in more detail in the PCT application WO 97/11151. The encapsulating material is typically selected from consisting of carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, polysaccharides, and combinations thereof. In some typical embodiments, the encapsulating material is a starch (see e.g., EP 0 922 499; U.S. Pat. No. 4,977,252; U.S. Pat. No. 5,354,559, and U.S. Pat. No. 5,935,826). In some embodiments, the encapsulating material is a microsphere made from plastic such as thermoplastics, acrylonitrile, methacrylonitrile, polyacrylonitrile, polymethacrylonitrile and mixtures thereof; commercially available microspheres that find use include, but are not limited to those supplied by EXPANCEL® (Stockviksverken, Sweden), and PM 6545, PM 6550, PM 7220, PM 7228, EXTENDOSPHERES®, LUXSIL®, Q-CEL®, and SPHERICEL® (PQ Corp., Valley Forge, Pa.).

In using detergent compositions that include TfuLip2 in cleaning applications, the fabrics, textiles, dishes, or other surfaces to be cleaned are incubated in the presence of the TfuLip2 detergent composition for a time sufficient to allow TfuLip2 to hydrolyze lipids present in soil or stains, and then typically rinsed with water or another aqueous solvent to remove the TfuLip2 detergent composition along with hydrolyzed lipids.

As described herein, the TfuLip2 polypeptides find particular use in the cleaning industry, including, but not limited to laundry and dish detergents. These applications place enzymes under various environmental stresses. The TfuLip2 polypeptides may provide advantages over many currently used enzymes, due to their stability under various conditions.

Indeed, there are a variety of wash conditions including varying detergent formulations, wash water volumes, wash water temperatures, and lengths of wash time, to which lipases involved in washing are exposed. In addition, detergent formulations used in different geographical areas have different concentrations of their relevant components present in the wash water. For example, European detergents typically have about 4,500-5,000 ppm of detergent components in the wash water, while Japanese detergents typically have approximately 667 ppm of detergent components in the wash water. In North America, particularly the United States, detergents typically have about 975 ppm of detergent components present in the wash water.

A low detergent concentration system includes detergents where less than about 800 ppm of the detergent components are present in the wash water. Japanese detergents are typically considered low detergent concentration system as they have approximately 667 ppm of detergent components present in the wash water.

A medium detergent concentration includes detergents where between about 800 ppm and about 2,000 ppm of the detergent components are present in the wash water. North

American detergents are generally considered to be medium detergent concentration systems as they have approximately 975 ppm of detergent components present in the wash water. Brazil typically has approximately 1,500 ppm of detergent components present in the wash water.

A high detergent concentration system includes detergents where greater than about 2000 ppm of the detergent components are present in the wash water. European detergents are generally considered to be high detergent concentration systems as they have approximately 4500-5000 ppm of detergent components in the wash water.

Latin American detergents are generally high suds phosphate builder detergents and the range of detergents used in Latin America can fall in both the medium and high detergent concentrations as they range from 1,500 ppm to 6000 ppm of detergent components in the wash water. As mentioned above, Brazil typically has approximately 1,500 ppm of detergent components present in the wash water. However, other high suds phosphate builder detergent geographies, not limited to other Latin American countries, may have high detergent concentration systems up to about 6,000 ppm of detergent components present in the wash water.

In light of the foregoing, it is evident that concentrations of detergent compositions in typical wash solutions throughout the world varies from less than about 800 ppm of detergent composition (“low detergent concentration geographies”), for example about 667 ppm in Japan, to between about 800 ppm to about 2,000 ppm (“medium detergent concentration geographies”), for example about 975 ppm in U.S. and about 1,500 ppm in Brazil, to greater than about 2,000 ppm (“high detergent concentration geographies”), for example about 4,500 ppm to about 5,000 ppm in Europe and about 6,000 ppm in high suds phosphate builder geographies.

The concentrations of the typical wash solutions are determined empirically. For example, in the U.S., a typical washing machine holds a volume of about 64.4 L of wash solution. Accordingly, in order to obtain a concentration of about 975 ppm of detergent within the wash solution about 62.79 g of detergent composition must be added to the 64.4 L of wash solution. This amount is the typical amount measured into the wash water by the consumer using the measuring cup provided with the detergent.

As a further example, different geographies use different wash temperatures. The temperature of the wash water in Japan is typically less than that used in Europe. For example, the temperature of the wash water in North America and Japan is typically between about 10 and about 30° C. (e.g., about 20° C.), whereas the temperature of wash water in Europe is typically between about 30 and about 60° C. (e.g., about 40° C.). However, in the interest of saving energy, many consumers are switching to using cold water washing. In addition, in some further regions, cold water is typically used for laundry, as well as dish washing applications. In some embodiments, the “cold water washing” of the present disclosure utilizes washing at temperatures from about 10° C. to about 40° C., or from about 20° C. to about 30° C., or from about 15° C. to about 25° C., as well as all other combinations within the range of about 15° C. to about 35° C., and all ranges within 10° C. to 40° C.

As a further example, different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million (parts per million converted to grains per U.S. gallon is ppm # divided by 17.1 equals grains per gallon) of hardness minerals.

TABLE II
Water Hardness Levels

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg²⁺.

Accordingly, in some embodiments, the present disclosure provides TfuLip2 polypeptides that show surprising wash performance in at least one set of wash conditions (e.g., water temperature, water hardness, and/or detergent concentration). In some embodiments, the TfuLip2 polypeptides are comparable in wash performance to other lipases. In some embodiments, the TfuLip2 polypeptides exhibit enhanced wash performance as compared to lipases currently commercially available. Thus, in some preferred embodiments, the TfuLip2 polypeptides provided herein exhibit enhanced oxidative stability, enhanced thermal stability, enhanced cleaning capabilities under various conditions, and/or enhanced chelator stability. In addition, the TfuLip2 polypeptides may find use in cleaning compositions that do not include detergents, again either alone or in combination with builders and stabilizers.

In some embodiments of the present disclosure, the cleaning compositions comprise at least one TfuLip2 polypeptide of the present disclosure at a level from about 0.00001% to about 10% by weight of the composition and the balance (e.g., about 99.999% to about 90.0%) comprising cleaning adjunct materials by weight of composition. In other aspects of the present disclosure, the cleaning compositions comprises at least one TfuLip2 polypeptide at a level of about 0.0001% to about 10%, about 0.001% to about 5%, about 0.001% to about 2%, about 0.005% to about 0.5% by weight of the composition and the balance of the cleaning composition (e.g., about 99.9999% to about 90.0%, about 99.999% to about 98%, about 99.995% to about 99.5% by weight) comprising cleaning adjunct materials.

In some embodiments, the cleaning compositions described herein comprise one or more additional detergent enzymes, which provide cleaning performance and/or fabric care and/or dishwashing benefits. Examples of suitable enzymes include, but are not limited to, hemicellulases, cellulases, peroxidases, proteases, xylanases, lipases, phospholipases, esterases, cutinases, pectinases, pectate lyases, mannanases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. In some embodiments, a combination of enzymes is used (i.e., a “cocktail”) comprising conventional applicable enzymes like protease, lipase, cutinase and/or cellulase in conjunction with amylase is used.

In addition to the TfuLip2 polypeptides provided herein, any other suitable lipase finds use in the compositions of the present disclosure. Suitable lipases include, but are not limited to those of bacterial or fungal origin. Chemically or genetically modified mutants are encompassed by the present disclosure. Examples of useful lipases include Humicola lanuginosa lipase (See e.g., EP 258 068, and EP 305 216), Rhizomucor miehei lipase (see e.g., EP 238 023), Candida lipase, such as C. antarctica lipase (e.g., the C. antarctica lipase A or B; See e.g., EP 214 761), Pseudomonas lipases such as P. alcaligenes lipase and P. pseudoalcaligenes lipase (see e.g., EP 218 272), P. cepacia lipase (see e.g., EP 331 376), P. stutzeri lipase (see e.g., GB 1,372,034), P. fluorescens lipase, Bacillus lipase (e.g., B. subtilis lipase; Dartois et al., Biochem. Biophys. Acta 1131:253-260, 1993); B. stearothermophilus lipase (see e.g., JP 64/744992); and B. pumilus lipase (see e.g., WO 91/16422).

Furthermore, a number of cloned lipases find use in some embodiments of the present disclosure, including but not limited to Penicillium camembertii lipase (see, Yamaguchi et al., Gene 103:61-67, 1991), Geotricum candidum lipase (see, Schimada et al., J. Biochem., 106:383-388, 1989), and various Rhizopus lipases such as R. delemar lipase (see, Hass et al., Gene 109:117-113, 1991), a R. niveus lipase (Kugimiya et al., Biosci. Biotech. Biochem. 56:716-719, 1992) and R. oryzae lipase.

Other types of lipolytic enzymes such as cutinases also find use in some embodiments of the present disclosure, including but not limited to the cutinase derived from Pseudomonas mendocina (see, WO 88/09367), and the cutinase derived from Fusarium solani pisi (see, WO 90/09446).

Additional suitable lipases include commercially available lipases such as M1 LIPASE™, LUMA FAST™, and LIPOMAX™ (Genencor); LIPOLASE® and LIPOLASE® ULTRA (Novozymes); and LIPASE P™ “Amano” (Amano Pharmaceutical Co. Ltd., Japan).

In some embodiments of the present disclosure, the cleaning compositions of the present disclosure further comprise lipases at a level from about 0.00001% to about 10% of additional lipase by weight of the composition and the balance of cleaning adjunct materials by weight of composition. In other aspects of the present disclosure, the cleaning compositions of the present disclosure also comprise lipases at a level of about 0.0001% to about 10%, about 0.001% to about 5%, about 0.001% to about 2%, about 0.005% to about 0.5% lipase by weight of the composition.

In some embodiments of the present disclosure, any suitable protease may be used. Suitable proteases include those of animal, vegetable or microbial origin. In some embodiments, chemically or genetically modified mutants are included. In some embodiments, the protease is a serine protease, preferably an alkaline microbial protease or a trypsin-like protease. In some embodiments, the protease is a subtilisin protease, including any of the large number of engineered subtilisin proteases known in the art. Various proteases are described in WO95/23221, WO 92/21760, U.S. Pat. Publ. No. 2008/0090747, and U.S. Pat. Nos. 5,801,039, 5,340,735, 5,500,364, 5,855,625, U.S. RE 34,606, 5,955,340, 5,700,676, 6,312,936, and 6,482,628, and various other patents. In some further embodiments, metalloproteases find use in the present disclosure, including but not limited to the neutral metalloprotease described in WO 07/044,993.

In some embodiments of the present disclosure, any suitable amylase may be used.

In some embodiments, any amylase (e.g., alpha and/or beta) suitable for use in alkaline solutions also find use. Suitable amylases include, but are not limited to those of bacterial or fungal origin. Chemically or genetically modified mutants are included in some embodiments. Amylases that find use in the present disclosure, include, but are not limited to α-amylases obtained from B. licheniformis (see e.g., GB 1,296,839). Commercially available amylases that find use in the present disclosure include, but are not limited to DURAMYL®, TERMAMYL®, FUNGAMYL®, STAINZYME®, STAINZYME PLUS®, STAINZYME ULTRA®, and BAN™ (Novozymes), as well as POWERASE™, RAPIDASE® and MAXAMYL® P (Danisco US Inc., Genencor Division).

In some embodiments of the present disclosure, the disclosed cleaning compositions of further comprise amylases at a level from about 0.00001% to about 10% of additional amylase by weight of the composition and the balance of cleaning adjunct materials by weight of composition. In other aspects of the present disclosure, the cleaning compositions also comprise amylases at a level of about 0.0001% to about 10%, about 0.001% to about 5%, about 0.001% to about 2%, about 0.005% to about 0.5% amylase by weight of the composition.

In some further embodiments, any suitable cellulase finds used in the cleaning compositions of the present disclosure. Suitable cellulases include, but are not limited to those of bacterial or fungal origin. Chemically or genetically modified mutants are included in some embodiments. Suitable cellulases include, but are not limited to Humicola insolens cellulases (see e.g., U.S. Pat. No. 4,435,307). Especially suitable cellulases are the cellulases having color care benefits (see e.g., EP 0 495 257). Commercially available cellulases that find use in the present include, but are not limited to CELLUZYME®, CAREZYME® (Novozymes), and KAC-500(B)™ (Kao Corporation). In some embodiments, cellulases are incorporated as portions or fragments of mature wild-type or variant cellulases, wherein a portion of the N-terminus is deleted (see e.g., U.S. Pat. No. 5,874,276). In some embodiments, the cleaning compositions of the present disclosure further comprise cellulases at a level from about 0.00001% to about 10% of additional cellulase by weight of the composition and the balance of cleaning adjunct materials by weight of composition. In other aspects of the present disclosure, the cleaning compositions also comprise cellulases at a level of about 0.0001% to about 10%, about 0.001% to about 5%, about 0.001% to about 2%, about 0.005% to about 0.5% cellulase by weight of the composition.

Any mannanase suitable for use in detergent compositions also finds use in the present disclosure. Suitable mannanases include, but are not limited to those of bacterial or fungal origin. Chemically or genetically modified mutants are included in some embodiments. Various mannanases are known which find use in the present disclosure (see e.g., U.S. Pat. No. 6,566,114, U.S. Pat. No. 6,602,842, and U.S. Pat. No. 6,440,991, all of which are incorporated herein by reference). In some embodiments, the disclosed cleaning compositions further comprise mannanases at a level from about 0.00001% to about 10% of additional mannanase by weight of the composition and the balance of cleaning adjunct materials by weight of composition. In other aspects of the present disclosure, the cleaning compositions also comprise mannanases at a level of about 0.0001% to about 10%, about 0.001% to about 5%, about 0.001% to about 2%, about 0.005% to about 0.5% mannanase by weight of the composition.

In some embodiments, peroxidases are used in combination with hydrogen peroxide or a source thereof (e.g., a percarbonate, perborate or persulfate) in the compositions of the present disclosure. In some alternative embodiments, oxidases are used in combination with oxygen. Both types of enzymes are used for “solution bleaching” (i.e., to prevent transfer of a textile dye from a dyed fabric to another fabric when the fabrics are washed together in a wash liquor), preferably together with an enhancing agent (see e.g., WO 94/12621 and WO 95/01426). Suitable peroxidases/oxidases include, but are not limited to those of plant, bacterial or fungal origin. Chemically or genetically modified mutants are included in some embodiments. In some embodiments, the cleaning compositions of the present disclosure further comprise peroxidase and/or oxidase enzymes at a level from about 0.00001% to about 10% of additional peroxidase and/or oxidase by weight of the composition and the balance of cleaning adjunct materials by weight of composition. In other aspects of the present disclosure, the cleaning compositions also comprise, peroxidase and/or oxidase enzymes at a level of about 0.0001% to about 10%, about 0.001% to about 5%, about 0.001% to about 2%, about 0.005% to about 0.5% peroxidase and/or oxidase enzymes by weight of the composition.

In some embodiments, additional enzymes find use, including but not limited to perhydrolases (see e.g., WO 05/056782). In addition, in some particularly preferred embodiments, mixtures of the above mentioned enzymes are encompassed herein, in particular one or more additional protease, amylase, lipase, mannanase, and/or at least one cellulase. Indeed, it is contemplated that various mixtures of these enzymes will find use in the present disclosure. It is also contemplated that the varying levels of the TfuLip2 polypeptide(s) and one or more additional enzymes may both independently range to about 10%, the balance of the cleaning composition being cleaning adjunct materials. The specific selection of cleaning adjunct materials are readily made by considering the surface, item, or fabric to be cleaned, and the desired form of the composition for the cleaning conditions during use (e.g., through the wash detergent use).

Examples of suitable cleaning adjunct materials include, but are not limited to, surfactants, builders, bleaches, bleach activators, bleach catalysts, other enzymes, enzyme stabilizing systems, chelants, optical brighteners, soil release polymers, dye transfer agents, dye transfer inhibiting agents, catalytic materials, hydrogen peroxide, sources of hydrogen peroxide, preformed peracis, polymeric dispersing agents, clay soil removal agents, structure elasticizing agents, dispersants, suds suppressors, dyes, perfumes, colorants, filler salts, hydrotropes, photoactivators, fluorescers, fabric conditioners, fabric softeners, carriers, hydrotropes, processing aids, solvents, pigments, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents (see, e.g., U.S. Pat. Nos. 6,610,642, 6,605,458, 5,705,464, 5,710,115, 5,698,504, 5,695,679, 5,686,014 and 5,646,101, all of which are incorporated herein by reference). Embodiments of specific cleaning composition materials are exemplified in detail below. In embodiments in which the cleaning adjunct materials are not compatible with the disclosed TfuLip2 polypeptides in the cleaning compositions, then suitable methods of keeping the cleaning adjunct materials and the lipase(s) separated (i.e., not in contact with each other) until combination of the two components is appropriate are used. Such separation methods include any suitable method known in the art (e.g., gelcaps, encapsulation, tablets, physical separation, etc.).

In some preferred embodiments, an effective amount of one or more TfuLip2 polypeptide(s) provided herein are included in compositions useful for cleaning a variety of surfaces in need of stain removal. Such cleaning compositions include cleaning compositions for such applications as cleaning hard surfaces, fabrics, and dishes. Indeed, in some embodiments, the present disclosure provides fabric cleaning compositions, while in other embodiments, the present disclosure provides non-fabric cleaning compositions. Notably, the present disclosure also provides cleaning compositions suitable for personal care, including oral care (including dentrifices, toothpastes, mouthwashes, etc., as well as denture cleaning compositions), skin, and hair cleaning compositions. It is intended that the present disclosure encompass detergent compositions in any form (i.e., liquid, granular, bar, semi-solid, gels, emulsions, tablets, capsules, etc.).

By way of example, several cleaning compositions wherein the disclosed TfuLip2 polypeptides find use are described in greater detail below. In some embodiments in which the disclosed cleaning compositions are formulated as compositions suitable for use in laundry machine washing method(s), the compositions of the present disclosure preferably contain at least one surfactant and at least one builder compound, as well as one or more cleaning adjunct materials preferably selected from organic polymeric compounds, bleaching agents, additional enzymes, suds suppressors, dispersants, lime-soap dispersants, soil suspension and anti-redeposition agents and corrosion inhibitors. In some embodiments, laundry compositions also contain softening agents (i.e., as additional cleaning adjunct materials). The compositions of the present disclosure also find use detergent additive products in solid or liquid form. Such additive products are intended to supplement and/or boost the performance of conventional detergent compositions and can be added at any stage of the cleaning process. In some embodiments, the density of the laundry detergent compositions herein ranges from about 400 to about 1200 g/liter, while in other embodiments, it ranges from about 500 to about 950 g/liter of composition measured at 20° C.

In embodiments formulated as compositions for use in manual dishwashing methods, the compositions of the disclosure preferably contain at least one surfactant and preferably at least one additional cleaning adjunct material selected from organic polymeric compounds, suds enhancing agents, group II metal ions, solvents, hydrotropes, and additional enzymes.

In some embodiments, various cleaning compositions such as those provided in U.S. Pat. No. 6,605,458, find use with the TfuLip2 polypeptides of the present disclosure. Thus, in some embodiments, the compositions comprising at least one TfuLip2 polypeptide of the present disclosure is a compact granular fabric cleaning composition, while in other embodiments, the composition is a granular fabric cleaning composition useful in the laundering of colored fabrics, in further embodiments, the composition is a granular fabric cleaning composition which provides softening through the wash capacity, in additional embodiments, the composition is a heavy duty liquid fabric cleaning composition. In some embodiments, the compositions comprising at least one TfuLip2 polypeptide of the present disclosure are fabric cleaning compositions such as those described in U.S. Pat. Nos. 6,610,642 and 6,376,450. In addition, the TfuLip2 polypeptides of the present disclosure find use in granular laundry detergent compositions of particular utility under European or Japanese washing conditions (see e.g., U.S. Pat. No. 6,610,642).

In some alternative embodiments, the present disclosure provides hard surface cleaning compositions comprising at least one TfuLip2 polypeptide provided herein. Thus, in some embodiments, the compositions comprising at least one TfuLip2 polypeptide of the present disclosure is a hard surface cleaning composition such as those described in U.S. Pat. Nos. 6,610,642, 6,376,450, and 6,376,450.

In yet further embodiments, the present disclosure provides dishwashing compositions comprising at least one TfuLip2 polypeptide provided herein. Thus, in some embodiments, the compositions comprising at least one TfuLip2 polypeptide of the present disclosure is a hard surface cleaning composition such as those in U.S. Pat. Nos. 6,610,642 and 6,376,450. In some still further embodiments, the present disclosure provides dishwashing compositions comprising at least one TfuLip2 polypeptide provided herein. In some further embodiments, the compositions comprising at least one TfuLip2 polypeptide of the present disclosure comprise oral care compositions such as those in U.S. Pat. Nos. 6,376,450, and 6,376,450. The formulations and descriptions of the compounds and cleaning adjunct materials contained in the aforementioned U.S. Pat. Nos. 6,376,450; 6,605,458; 6,605,458; and 6,610,642, find use with the TfuLip2 polypeptides provided herein.

The cleaning compositions of the present disclosure are formulated into any suitable form and prepared by any process chosen by the formulator, non-limiting examples of which are described in U.S. Pat. Nos. 5,879,584; 5,691,297; 5,574,005; 5,569,645; 5,565,422; 5,516,448; 5,489,392; and 5,486,303, all of which are incorporated herein by reference. When a low pH cleaning composition is desired, the pH of such composition is adjusted via the addition of a material such as monoethanolamine or an acidic material such as HCl.

While not essential for the purposes of the present disclosure, the non-limiting list of adjuncts illustrated hereinafter are suitable for use in the instant cleaning compositions. In some embodiments, these adjuncts are incorporated for example, to assist or enhance cleaning performance, for treatment of the substrate to be cleaned, or to modify the aesthetics of the cleaning composition as is the case with perfumes, colorants, dyes or the like. It is understood that such adjuncts are in addition to the TfuLip2 polypeptides of the present disclosure. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, deposition aids, dispersants, additional enzymes, and enzyme stabilizers, catalytic materials, bleach activators, bleach boosters, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments. In addition to the disclosure below, suitable examples of such other adjuncts and levels of use are found in U.S. Pat. Nos. 5,576,282; 6,306,812; and 6,326,348, incorporated by reference. The aforementioned adjunct ingredients may constitute the balance of the cleaning compositions of the present disclosure.

In some embodiments, the cleaning compositions according to the present disclosure comprise at least one surfactant and/or a surfactant system wherein the surfactant is selected from nonionic surfactants, anionic surfactants, cationic surfactants, ampholytic surfactants, zwitterionic surfactants, semi-polar nonionic surfactants and mixtures thereof. In some low pH cleaning composition embodiments (e.g., compositions having a neat pH of from about 3 to about 5), the composition typically does not contain alkyl ethoxylated sulfate, as it is believed that such surfactant may be hydrolyzed by such compositions the acidic contents. In some embodiments, the surfactant is present at a level of from about 0.1% to about 60%, while in alternative embodiments the level is from about 1% to about 50%, while in still further embodiments the level is from about 5% to about 40%, by weight of the cleaning composition.

In some embodiments, the cleaning compositions of the present disclosure contain at least one chelating agent. Suitable chelating agents may include, but are not limited to copper, iron and/or manganese chelating agents and mixtures thereof. In embodiments in which at least one chelating agent is used, the cleaning compositions of the present disclosure comprise from about 0.1% to about 15% or even from about 3.0% to about 10% chelating agent by weight of the subject cleaning composition.

In some still further embodiments, the cleaning compositions provided herein contain at least one deposition aid. Suitable deposition aids include, but are not limited to, polyethylene glycol, polypropylene glycol, polycarboxylate, soil release polymers such as polytelephthalic acid, clays such as kaolinite, montmorillonite, atapulgite, illite, bentonite, halloysite, and mixtures thereof.

As indicated herein, in some embodiments, anti-redeposition agents find use in some embodiments of the present disclosure. In some preferred embodiments, non-ionic surfactants find use. For example, in automatic dishwashing embodiments, non-ionic surfactants find use for surface modification purposes, in particular for sheeting, to avoid filming and spotting and to improve shine. These non-ionic surfactants also find use in preventing the re-deposition of soils. In some preferred embodiments, the anti-redeposition agent is a non-ionic surfactant as known in the art (see e.g., EP 2 100 949).

In some embodiments, the cleaning compositions of the present disclosure include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. In embodiments in which at least one dye transfer inhibiting agent is used, the cleaning compositions of the present disclosure comprise from about 0.0001% to about 10%, from about 0.01% to about 5%, or even from about 0.1% to about 3% by weight of the cleaning composition.

In some embodiments, silicates are included within the compositions of the present disclosure. In some such embodiments, sodium silicates (e.g., sodium disilicate, sodium metasilicate, and crystalline phyllosilicates) find use. In some embodiments, silicates are present at a level of from about 1% to about 20%. In some preferred embodiments, silicates are present at a level of from about 5% to about 15% by weight of the composition.

In some still additional embodiments, the cleaning compositions of the present disclosure also contain dispersants. Suitable water-soluble organic materials include, but are not limited to the homo- or co-polymeric acids or their salts, in which the polycarboxylic acid comprises at least two carboxyl radicals separated from each other by not more than two carbon atoms.

In some further embodiments, the enzymes used in the cleaning compositions are stabilized any suitable technique. In some embodiments, the enzymes employed herein are stabilized by the presence of water-soluble sources of calcium and/or magnesium ions in the finished compositions that provide such ions to the enzymes. In some embodiments, the enzyme stabilizers include oligosaccharides, polysaccharides, and inorganic divalent metal salts, including alkaline earth metals, such as calcium salts. It is contemplated that various techniques for enzyme stabilization will find use in the present disclosure. For example, in some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), nickel (II), and oxovanadium (IV). Chlorides and sulfates also find use in some embodiments of the present disclosure. Examples of suitable oligosaccharides and polysaccharides (e.g., dextrins) are known in the art (see e.g., WO 07/145,964). In some embodiments, reversible protease inhibitors also find use, such as boron-containing compounds (e.g., borate, 4-formyl phenyl boronic acid) and/or a tripeptide aldehyde find use to further improve stability, as desired.

In some embodiments, bleaches, bleach activators and/or bleach catalysts are present in the compositions of the present disclosure. In some embodiments, the cleaning compositions of the present disclosure comprise inorganic and/or organic bleaching compound(s). Inorganic bleaches may include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Any suitable salt known in the art finds use in the present disclosure (see e.g., EP 2 100 949).

In some embodiments, bleach activators are used in the compositions of the present disclosure. Bleach activators are typically organic peracid precursors that enhance the bleaching action in the course of cleaning at temperatures of 60° C. and below. Bleach activators suitable for use herein include compounds which, under perhydrolysis conditions, give aliphaic peroxoycarboxylic acids having preferably from about 1 to about 10 carbon atoms, in particular from about 2 to about 4 carbon atoms, and/or optionally substituted perbenzoic acid. Additional bleach activators are known in the art and find use in the present disclosure (see e.g., EP 2 100 949).

In addition, in some embodiments and as further described herein, the cleaning compositions of the present disclosure further comprise at least one bleach catalyst. In some embodiments, the manganese triazacyclononane and related complexes find use, as well as cobalt, copper, manganese, and iron complexes. Additional bleach catalysts find use in the present disclosure (see e.g., U.S. Pat. Nos. 4,246,612, 5,227,084, 4,810410, WO 99/06521, and EP 2 100 949).

In some embodiments, the cleaning compositions of the present disclosure contain one or more catalytic metal complexes. In some embodiments, a metal-containing bleach catalyst finds use. In some preferred embodiments, the metal bleach catalyst comprises a catalyst system comprising a transition metal cation of defined bleach catalytic activity, (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (see e.g., U.S. Pat. No. 4,430,243). In some embodiments, the cleaning compositions of the present disclosure are catalyzed by means of a manganese compound. Such compounds and levels of use are well known in the art (See e.g., U.S. Pat. No. 5,576,282). In additional embodiments, cobalt bleach catalysts find use in the cleaning compositions of the present disclosure. Various cobalt bleach catalysts are known in the art (see e.g., U.S. Pat. Nos. 5,597,936 and 5,595,967) and are readily prepared by known procedures.

In some additional embodiments, the cleaning compositions of the present disclosure include a transition metal complex of a macropolycyclic rigid ligand (MRL). As a practical matter, and not by way of limitation, in some embodiments, the compositions and cleaning processes provided by the present disclosure are adjusted to provide on the order of at least one part per hundred million of the active MRL species in the aqueous washing medium, and in some preferred embodiments, provide from about 0.005 ppm to about 25 ppm, more preferably from about 0.05 ppm to about 10 ppm, and most preferably from about 0.1 ppm to about 5 ppm, of the MRL in the wash liquor.

In some embodiments, preferred transition-metals in the instant transition-metal bleach catalyst include, but are not limited to manganese, iron and chromium. Preferred MRLs also include, but are not limited to special ultra-rigid ligands that are cross-bridged (e.g., 5,12-diethyl-1,5,8,12-tetraazabicyclo[6.6.2]hexadecane). Suitable transition metal MRLs are readily prepared by known procedures (see e.g., WO 2000/32601, and U.S. Pat. No. 6,225,464).

In some embodiments, the cleaning compositions of the present disclosure comprise metal care agents. Metal care agents find use in preventing and/or reducing the tarnishing, corrosion, and/or oxidation of metals, including aluminum, stainless steel, and non-ferrous metals (e.g., silver and copper). Suitable metal care agents include those described in EP 2 100 949, WO 9426860 and WO 94/26859). In some embodiments, the metal care agent is a zinc salt. In some further embodiments, the cleaning compositions of the present disclosure comprise from about 0.1% to about 5% by weight of one or more metal care agent.

As indicated above, the cleaning compositions of the present disclosure are formulated into any suitable form and prepared by any process chosen by the formulator, non-limiting examples of which are described in U.S. Pat. Nos. 5,879,584; 5,691,297; 5,574,005; 5,569,645; 5,516,448; 5,489,392; and 5,486,303, all of which are incorporated herein by reference. In some embodiments in which a low pH cleaning composition is desired, the pH of such composition is adjusted via the addition of an acidic material such as HCl.

The cleaning compositions disclosed herein of find use in cleaning a situs (e.g., a surface, dishware, or fabric). Typically, at least a portion of the situs is contacted with an embodiment of the present cleaning composition, in neat form or diluted in a wash liquor, and then the situs is optionally washed and/or rinsed. For purposes of the present disclosure, “washing” includes but is not limited to, scrubbing, and mechanical agitation. In some embodiments, the cleaning compositions are typically employed at concentrations of from about 500 ppm to about 15,000 ppm in solution. When the wash solvent is water, the water temperature typically ranges from about 5° C. to about 90° C. and, when the situs comprises a fabric, the water to fabric mass ratio is typically from about 1:1 to about 30:1.

### VI. TfuLip2 Polypeptides as Chemical Reagents

The preference of TfuLip2 for short-chain lipids make the present polypeptides particularly useful for performing transesterification reactions involving C4-C16 substrates. Exemplary applications are the hydrolysis of milk fat; the synthesis of structured triglycerides, the synthesis and degradation of polymers, the formation of emulsifying agents and surfactants; the synthesis of ingredients for personal-care products, pharmaceuticals and agrochemicals, for making esters for use as perfumes and fragrances, for making biofuels and synthetic lubricants, for forming peracids, and for other uses in the oleochemical industry. Further uses for the above-described enzyme are described in U.S. Patent Pubs. 20070026106; 20060078648; and 20050196766, and in WO 2005/066347, which documents are incorporated by reference.

In general terms, a substrate and acceptor molecule are incubated in the presence of an TfuLip2 polypeptide or variant thereof under conditions suitable for performing a transesterification reaction, followed by, optionally, isolating a product from the reaction. Alternatively, the conditions may in the context of a foodstuff and the product may become a component of the foodstuff without isolation.

Other aspects and embodiments of the present compositions and methods will apparent from the foregoing description and following examples.

### EXAMPLES

The following examples are provided to demonstrate and illustrate certain preferred embodiments and aspects of the present disclosure and should not be construed as limiting.

In the experimental disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml and mL (milliliters); μl and μL (microliters); cm (centimeters); mm (millimeters); μm (micrometers); nm (nanometers); U (units); MW (molecular weight); sec (seconds); min(s) (minute/minutes); h(s) and hr(s) (hour/hours); ° C. (degrees Centigrade); QS (quantity sufficient); ND (not done); rpm (revolutions per minute); H₂O (water); dH₂O (deionized water); (HCl (hydrochloric acid); aa (amino acid); bp (base pair); kb (kilobase pair); kD (kilodaltons); MgCl₂ (magnesium chloride); NaCl (sodium chloride); w/v (weight to volume); v/v (volume to volume); g (gravity); OD (optical density); ppm (parts per million); m- (meta-); o- (ortho-); p- (para-); BCE (BCE103 cellulase); Glu-BL (Bacillus licheniformis glutamyl endopeptidase I); TfuLip2 (Thermobifida fusca lipase2); NEFA (non-esterified fatty acids); p-NP (para-nitrophenyl); SRI (stain removal index).

### Example 1

### Cloning and Expression of Thermobifida fusca Lipase2 (TfuLip2)

The Thermobifida fusca lipase 2 (or BTA-hydrolase 2) gene was previously identified (Lykidis et al., J. Bacteriol, 189:2477-2486, 2007), with the sequence set forth as GENBANK Accession No. YP_{—}288944. The B. subtilis expression vector p2JM103BBI (Vogtentanz, Protein Expr Purif, 55:40-52, 2007) was digested with the restriction enzymes BssHII and HindIII. The DNA fragment devoid of the BCE103-BBI fusion gene sequence was isolated and used as the expression backbone. Ligation of this fragment to a synthetic gene encoding a TfuLip2 enzyme resulted in the fusion of the N-terminus of the TfuLip2 polypeptide to the third amino acid of the Bacillus subtilis AprE pro-peptide encoded by p2JM103BBI. Following the natural signal peptidase cleavage in the host, the recombinant TfuLip2 protein produced in this manner has three additional amino acids (Ala-Gly-Lys) at its amino-terminus.

The nucleotide sequence of the Thermobifida fusca lipase2 (TfuLip2) synthetic gene is set forth as SEQ ID NO: 1:

GCGCGCAGGCTGCTGGAAAAGCTAATCCTTACGAAAGAGGACCGAATCCT
ACAGACGCGCTTCTGGAGGCTTCAAGCGGACCTTTTTCTGTTTCTGAAGA
AAACGTTTCTAGACTTAGCGCGTCTGGCTTTGGTGGCGGGACAATTTATT
ACCCGAGAGAGAATAACACATACGGGGCGGTGGCAATCTCTCCGGGGTAC
ACGGGCACAGAAGCATCTATTGCTTGGCTTGGTGAAAGAATTGCTTCTCA
TGGCTTTGTTGTAATCACAATTGACACAATTACGACACTTGATCAACCGG
ATTCAAGAGCTGAACAATTGAATGCAGCCCTGAATCATATGATCAACAGA
GCTTCAAGCACGGTAAGAAGCAGAATTGATAGCTCAAGACTGGCGGTGAT
GGGACATAGCATGGGAGGCGGAGGCACACTTAGATTAGCCTCACAGAGAC
CTGATTTAAAGGCAGCGATTCCGTTGACGCCTTGGCATCTGAACAAAAAT
TGGTCTAGCGTGACAGTCCCGACGCTCATTATCGGAGCAGATCTCGATAC
GATTGCACCGGTCGCGACACATGCCAAACCGTTCTATAACTCATTGCCGA
GCTCAATCTCAAAAGCCTATTTAGAACTGGATGGCGCCACACATTTTGCG
CCGAATATTCCGAACAAGATTATCGGTAAATATTCAGTCGCATGGTTAAA
AAGATTTGTAGATAATGACACGAGATATACGCAGTTCCTGTGTCCTGGGC
CTAGAGACGGTTTGTTCGGAGAGGTTGAAGAGTATAGAAGCACGTGCCCG
TTTTAAAAGCTT

The amino acid sequence of the mature TfuLip2 enzyme is set forth as SEQ ID NO: 2:

ANPYERGPNPTDALLEASSGPFSVSEENVSRLSASGFGGGTIYYPRENNT
YGAVAISPGYTGTEASIAWLGERIASHGFVVITIDTITTLDQPDSRAEQL
NAALNHMINRASSTVRSRIDSSRLAVMGHSMGGGGTLRLASQRPDLKAAI
PLTPWHLNKNWSSVTVPTLIIGADLDTIAPVATHAKPFYNSLPSSISKAY
LELDGATHFAPNIPNKIIGKYSVAWLKRFVDNDTRYTQFLCPGPRDGLFG
EVEEYRSTCPF

The amino acid sequence of the TfuLip2 enzyme with a three amino acid amino-terminal extension is set forth as SEQ ID NO: 3:

AGKANPYERGPNPTDALLEASSGPFSVSEENVSRLSASGFGGGTIYYPRE
NNTYGAVAISPGYTGTEASIAWLGERIASHGFVVITIDTITTLDQPDSRA
EQLNAALNHMINRASSTVRSRIDSSRLAVMGHSMGGGGTLRLASQRPDLK
AAIPLTPWHLNKNWSSVTVPTLIIGADLDTIAPVATHAKPFYNSLPSSIS
KAYLELDGATHFAPNIPNKIIGKYSVAWLKRFVDNDTRYTQFLCPGPRDG
LFGEVEEYRSTCPF

The TfuLip2 protein was produced in Bacillus subtilis cells (degU^{Hy}32, oppA, ΔspoIIE, ΔaprE, ΔnprE, Δepr, ΔispA, Δbpr, Δvpr, ΔwprA, Δmpr-ybfJ, ΔnprB, amyE::xylRPxylAcomK-ermC) using previously described methods (Vogtentanz, Protein Expr Purif, 55:40-52, 2007).

### Example 2

### TfuLip2 Isolation and Characterization

Ultra-filtered concentrate was derived from a 14-L scale batch fermentation of the expression Bacillus subtilis strain. The clarified broth was used for characterization of the recombinant TfuLip2 polypeptide.

For purification of TfuLip2, ultra-filtered concentrate is derived from a 14-L scale batch fermentation and is diluted 5-fold with 50 mM Tris-HCl, pH 8.0, buffer, and ammonium sulfate is added to a final concentration of 1 M. The pellet from the ammonium sulfate precipitation is collected and used for further purification. A FastFlow Phenyl Sepharose column equilibrated with 1 M ammonium sulfate in 50 mM Tris-HCl, pH 8.0, buffer is used. Sample is loaded at half the equilibration flow rate (12 ml/min) and washed with equilibration buffer after loading. A gradient is used to reduce the concentration from 1 M ammonium sulfate to 0 M, in buffer. Contaminant proteins are washed off the column with the 50 mM Tris, pH 8.0, buffer. The TfuLip2 protein is eluted with a buffer containing 50 mM Tris HCl, pH 8.0, and 40% propylene glycol. Fractions are assayed using the para-nitrophenyl (pNP) butyrate assay described below. Fractions containing lipase activity are pooled and concentrated using a stir cell with a 5K membrane in preparation for subsequent use.

### Example 3

### Hydrolysis of p-Nitrophenyl Esters by TfuLip2

The TfuLip2 protein was assayed for lipase activity on three different para-nitrophenyl (pNP) ester substrates with varying ester chain lengths to determine the chain length preference of LipA. Table 3-1 provides details of the pNP ester substrates.

TABLE 3-1
pNP Ester Substrates

| Substrate | Abbr | Chain-length | Source |
|---|---|---|---|
| p-nitrophenyl butyrate | pNB | C4:0 | Sigma (CAS 2635-84-9) |
| p-nitrophenyl caprylate | pNO | C8:0 | Sigma (CAS 1956-10-1) |
| p-nitrophenyl palmitate | pNP | C16:0 | Sigma (CAS 1492-30-4) |

A reaction emulsion with pNP ester substrates was prepared using 0.8 mM pNP ester pre-suspended in ethanol (5%) in one of two buffers: 0.05 M HEPES, 6 mM CaCl₂, adjusted to pH 8.2, or 0.05 M CAPS, 6 mM CaCl₂, adjusted to pH 10. To aid in the emulsification of the pNP-esters, 0.5% gum Arabic was added to both buffers.

The pNP-ester/buffer suspensions were mixed, ultra-sonicated for 2 minutes and 100 μL of each was transferred to 96-well microtiter plate wells containing 20 μL enzyme samples. The generation of liberated pNP was monitored over a period of 15 minutes at OD₄₀₅ nm and corrected using blank values (no enzyme). The pNP product generated per minute was recorded and normalized to the added enzyme sample in the well (delta OD/min per added mg enzyme). The relative enzyme activity on the different substrates was calculated, and the rate of product release obtained using each substrate was normalized to the highest activity (e.g., activity on the pNP-caprylate substrate was set to 100).

TABLE 3-2
Chain Length Preference of TfuLip2

| | pH 8.2 | | | pH 10 | |
|---|---|---|---|---|---|
| | Butyrate | Caprylate | Palmitate | Caprylate | Palmitate |
| TfuLip2 | 30 | 100 | 50 | 100 | 80 |

As shown in Table 3-2, TfuLip2 shows activity towards pNP-ester substrates from 4 to 16 carbons long, at both pH 8.2 and 10.

### Example 4

### Triglyceride Hydrolysis by TfuLip2 in the Presence and Absence of Detergent

TfuLip2 polypeptide was assayed for hydrolysis of trioctanoate and trioleate substrates in the presence and absence of a detergent. The glyceryl trioctanoate (CAS 538-23-8) and glyceryl trioleate (CAS 122-32-7) substrates were purchased from Sigma. The following commercially available detergents were used for this experiment: (1) OMO color, liquid detergent, from Unilever; (2) Ariel color, liquid detergent, from Procter & Gamble; (3) Biotex color, powder detergent, from Blumøller; and (4) Ariel color, powder detergent, from Procter & Gamble.

### OMO Color Liquid Detergent

The OMO color liquid detergent composition comprises 5-15% anionic surfactants and nonionic surfactants, <5% soap, cationic surfactants, phosphonates, perfume, butylphenyl methylptopionate, citronellol, enzymes, and benzisothiazolinone. The OMO color liquid detergent contains the following surfactants: C12-C15 pareth-7, sodium dodecylbenzene sulfonate, sodium laureth sulfate, and sodium hydrogenated cocoate.

Ingredients of the OMO color liquid detergent are as follows: water, C12-C15 pareth-7, sodium dodecylbenzene sulfonate, sodium laureth sulfate, propylene glycol, sodium hydrogenated cocoate, sodium diethylenetriamine pentamethylene phosphonate, perfume, sodium sulfate, sodium hydroxide, butylphenyl methylpropional, sorbitol, citronellol, protease, benzisothiazolinone, boronic acid, (4-formylphenyl), amylase, CI-45100, and CI 42051.

### Ariel Color Liquid Detergent

The Ariel color liquid detergent composition comprises 5-15% anionic surfactants, <5% nonionic surfactants, phosphonates, soap, enzymes, perfume, butylphenyl methylptopionate, and geraniol. The Ariel color liquid detergent contains the following surfactants: sodium dodecylbenzene sulfonate, C12-C14 pareth-7, sodium laureth sulfate, and C12-C14 pareth-4.

Ingredients of the Ariel color liquid detergent are as follows: sodium dodecylbenzene sulfonate, sodium citrate, sodium palm kernelate, C12-C14 pareth-7, sodium laureth sulfate, alcohol denatured, C14-C15 pareth-4, mea-borate, sulfated ethoxylated hexamethylenediamine quaternized, propylene glycol, water, hydrogenated castor oil, parfum, protease, sodium diethylenetriamine pentamethylene phosphonate, C12-C15 alcohols, glycosidase, polyvinylpyridine-n-oxide, polyethylene glycol, sodium sulfate, sodium chloride, dimethicone, colorant, silica, butylphenyl methylpropional, and geraniol.

### Biotex Color Powder Detergent

The Biotex color powder detergent composition comprises 15-30% zeolite, 5-15% anionic surfactants, <5% soap, polycarboxylates, phosphonates, enzymes, and perfume. The Biotex color powder detergent contains the C12-C15 pareth-7 surfactant.

Ingredients of the Biotex color liquid detergent are as follows: zeolite, sodium carbonate, sodium sulfate, water, C12-C15 pareth-7, sodium tallowate, maleic acid-acrylic acid copolymer sodium salt, sodium citrate, laureth-7, cellulose gum, laureth-5, sodium EDTMP, parfum, tetrasodium etidronate, subtilisin, amylase, triacylglycerol lipase, and cellulase.

### Ariel Color Powder Detergent

The Ariel color powder detergent composition comprises 5-15% anionic surfactants, zeolite, <5% nonionic surfactants, polycarboxylates, phosphonates, enzymes, perfume, hexyl cinnamal, limonene, and butylphenyl methylptopionate. The Ariel color powder detergent contains the following surfactants: sodium dodecylbenzene sulfonate, sodium C12-C15 pareth sulfate, and C12-C15 pareth-7.

Ingredients of the Ariel color powder detergent are as follows: sodium sulfate, sodium carbonate, bentonite, sodium dodecylbenzene sulfonate, sodium silicoaluminate, sodium C12-C15 pareth sulfate, sodium acrylic acid/MA copolymer, water, citric acid, dimethicone, C12-C15 pareth-7, magnesium sulfate, sodium dodecylbenzene sulfonate, parfum, cellulose gum, sodium chloride, tetrasodium etidronate, sodium toluenesulfonate, starch, sodium octenyl succinate, polyethylene glycol, glycosidase, trisodium ethylenediamine disuccinate, sulfuric acid, sodium glycollate, phenylpropyl ether methicone, sodium polyacrylate, dodecylbenzene sulfonic acid, dichlorodimethylsilane RX with silica, colorant, glycerine, sodium laureth sulfate, sodium hydroxide, C10-16 alkylbenzene sulfonic acid, butylphenyl methylpropional, hexyl cinnamal, and linalool.

The detergents were heat-inactivated as follows: the liquid detergents were placed in a water bath at 95° C. for 2 hours, while 0.1 g/mL preparations in water of the powder detergents were boiled on a hot plate for 1 hour. Heat treatments inactivate the enzymatic activity of any protein components in commercial detergent formulas, while retaining the properties of the non-enzymatic detergent components. Following heating, the detergents are diluted and assayed for lipase enzyme activity.

Reaction emulsion of trioctanoate and trioleate were prepared from 0.4% trioctanoate or trioleate pre-suspended in ethanol (5%), in one of 2 buffers: 0.05 M HEPES adjusted to pH 8.2, or 0.05 M CAPS adjusted to pH 10. The buffer was adjusted to pH 8.2 for use with liquid detergent, and to pH 10 for use with powder detergent. For both buffers water hardness was adjusted to 6 mM CaCl₂. 2% gum Arabic was added to both buffers to aid in the emulsification of the triglyceride.

Reaction emulsions of trioctanoate in each of the detergents was prepared from 0.4% trioctanoate pre-suspended in ethanol (5%), in one of two buffers: 0.05 M HEPES adjusted to pH 8.2, or 0.05 M CAPS adjusted to pH 10. For both buffers water hardness adjusted to 240 ppm. The final assay mixtures contained varying amounts of detergents, to aid in the emulsification of the triglyceride.

The reaction emulsions were made by applying high shear mixing for 2 minutes (24,000 m⁻¹, Ultra Turrax T25, Janke & Kunkel), and then transferring 150 μL to 96-well microtiter plate wells already containing 30 μL enzyme samples. Free fatty acid generation was measured using an in vitro enzymatic colorimetric assay for the quantitative determination of non-esterified fatty acids (NEFA). This method is specific for free fatty acids, and relies upon the acylation of coenzyme A (CoA) by the fatty acids in the presence of added acyl-CoA synthetase. The acyl-CoA thus produced is oxidized by added acyl-CoA oxidase with generation of hydrogen peroxide, in the presence of peroxidase. This permits the oxidative condensation of 3-methyl-N-ethyl-N(β-hydroxyethyl)-aniline with 4-aminoantipyrine to form a purple colored adduct which can be measured colorimetrically. The amount of free fatty acids generated after a 6 minute incubation at 30° C. was determined using the materials in a NEFA HR(2) kit (Wako Chemicals GmbH, Germany) by transferring 30 μL of the hydrolysis solution to 96-well microtiter plate wells already containing 120 μL NEFA A solution. Incubation for 3 min at 30° C. was followed by addition of 60 μL NEFA B solution. After incubation for 4.5 min at 30° C. OD at 520 nm was measured.

Table 4-1 shows hydrolysis of trioleate and trioctanoate by TfuLip2. Data for triglyceride hydrolysis was determined as μmol free fatty acid. The results are reported relative to the activity on trioctanoate (C8) in buffer, which was set to 100.

TABLE 4-1
Trioleate and Trioctanoate Hydrolysis by TfuLip2 in Buffer

| pH 8.2 | | pH 10 | |
|---|---|---|---|
| Trioctanoate | Trioleate | Trioctanoate | Trioleate |

| | | | | |
|---|---|---|---|---|
| TfuLip2 | 100 | 11 | 100 | 11 |

Table 4-2 shows trioctanoate hydrolysis by TfuLip2 in the presence or absence of various detergents at pH 8.2 and pH 10.0. Data for trioctanoate hydrolysis in the presence of detergent is reported as percent trioctanoate hydrolysis in the presence of detergent relative to trioctanoate hydrolysis in the absence of detergent at both pH values tested.

TABLE 4-2
Trioctanoate Hydrolysis by TfuLip2 in Detergent Compositions

| | No | 1 ml OMO | 2.6 ml OMO | 1 ml Ariel | 2.5 mL Ariel |
|---|---|---|---|---|---|
| pH | detergent | liquid/L | liquid/L | liquid/L | liquid/L |
| 8.2 | 100 | 40 | 6 | 60 | 60 |
| | No | 1 g Ariel | 1.7 g Ariel | 1 g BioTex | 2.2 g Biotex |
| pH | detergent | powder/L | powder/L | powder/L | powder/L |
| 10 | 100 | 70 | 34 | 23 | 10 |

TfuLip2 shows lipase activity in various liquid and powder detergents as a function of detergent concentration.

### Example 5

### Cleaning Performance of TfuLip2

Cleaning performance of TfuLip2 on stained fabrics was tested in a microswatch assay format. Stain removal experiments were carried out using a lipid-containing technical stain (CS-61 swatches, purchased from Center for Testmaterials, Netherlands) set in a 24-well plate format (Nunc, Denmark) Each assay well was set to contain a pre-cut 13 mm piece of CS-61 swatch. Swatches were pre-read using a reflectometer (CR-400, Konica Minolta) before placement in the 24-well plate.

The buffers used were 20 mM HEPES (final concentration) pH 8.2 for testing liquid detergents, and 20 mM CAPS (final concentration) pH 10.0 for testing powder detergents. Water hardness was adjusted to 240 ppm for both buffers. The commercially available, heat-inactivated detergents used were the same as described in the triglyceride hydrolysis assay of Example 4.

Briefly, 900 μl of the appropriate buffer was added to each swatch-containing well of the 24-well plate. To initiate the reaction, enzyme samples were added at a volume of 100 μL into each well. The plates were shaken for 30 minutes at 200 rpm at 37° C. After incubation, the reaction buffer was removed and the fabric in each well was rinsed with 1 mL distilled water three times. After removing the rinsed swatches, the swatches were dried at 50° C. for 4 hours before reflectance was measured. Cleaning was calculated as the difference of the post- and pre-cleaning reflectometry measurements for each swatch. Measurement of reflectance was performed by taking CIE L*a*b* measurements with a spectrophotometer (CR-400, Konica Minolta). A difference in stain removal index (ΔSRI) values of the washed fabric were calculated in relation to the unwashed fabrics using the formula:

Total color difference(ΔSRI)=\sqrt{(ΔL²+Δa²+Δb²)}

In this equation, ΔL, Δa, Δb, are differences in CIE L*, CIE a*, and CIE b* values respectively before and after cleaning, where L* defines lightness and a* and b* define chromaticity (see, e.g., Precise Color Communication: Color Control From Perception to Instrumentation, Konica Minolta Sensing, Inc., Osaka, Japan, pp. 32-59, 1998).

TABLE 5-1
Cleaning Performance of TfuLip2 on Stained Fabric

| 0.4 g/L detergent | |
|---|---|
| Powder detergent | Liquid detergent |

| | Ariel | Biotex | Ariel | OMO |
|---|---|---|---|---|
| TfuLip2 | +++ | ++ | ++ | − |

TfuLip2 shows no cleaning performance in OMO Color liquid detergent from Unilever. However, TfuLip2 exhibited significant cleaning performance in Ariel Color liquid detergent from Procter & Gamble, and in Biotex Color powder detergent from Blumøller, with even greater performance in Ariel Color powder detergent from Procter & Gamble.

### Example 6

### Liquid Laundry Detergent Compositions Comprising TfuLip2

In this example, various formulations for liquid laundry detergent compositions are provided. In each of these formulations, TfuLip2 is included at a concentration of from about 0.0001 to about 10 weight-percent. In some alternative embodiments, other concentrations will find use, as determined by the formulator, based on their needs.

TABLE 6-1
Liquid Laundry Detergent Compositions

| | Formulations | | | | |
|---|---|---|---|---|---|
| Compound | I | II | III | IV | V |
| LAS | 24.0 | 32.0 | 6.0 | 3.0 | 6.0 |
| NaC₁₆-C₁₇ HSAS | — | — | — | 5.0 | — |
| C₁₂-C₁₅ AE_{1.8}S | — | — | 8.0 | 7.0 | 5.0 |
| C₈-C₁₀ propyl dimethyl | 2.0 | 2.0 | 2.0 | 2.0 | 1.0 |
| amine | | | | | |
| C₁₂-C₁₄ alkyl dimethyl | — | — | — | — | 2.0 |
| amine oxide | | | | | |
| C₁₂-C₁₅ AS | — | — | 17.0 | — | 8.0 |
| CFAA | — | 5.0 | 4.0 | 4.0 | 3.0 |
| C₁₂-C₁₄ Fatty alcohol | 12.0 | 6.0 | 1.0 | 1.0 | 1.0 |
| ethoxylate | | | | | |
| C₁₂-C₁₈ Fatty acid | 3.0 | — | 4.0 | 2.0 | 3.0 |
| Citric acid (anhydrous) | 4.5 | 5.0 | 3.0 | 2.0 | 1.0 |
| DETPMP | — | — | 1.0 | 1.0 | 0.5 |
| Monoethanolamine | 5.0 | 5.0 | 5.0 | 5.0 | 2.0 |
| Sodium hydroxide | — | — | 2.5 | 1.0 | 1.5 |
| 1N HCl aqueous solution | #1 | #1 | — | — | — |
| Propanediol | 12.7 | 14.5 | 13.1 | 10. | 8.0 |
| Ethanol | 1.8 | 2.4 | 4.7 | 5.4 | 1.0 |
| DTPA | 0.5 | 0.4 | 0.3 | 0.4 | 0.5 |
| Pectin Lyase | — | — | — | 0.005 | — |
| Amylase | 0.001 | 0.002 | — | | — |
| Cellulase | — | — | 0.0002 | | 0.0001 |
| Lipase | 0.1 | — | 0.1 | — | 0.1 |
| NprE (optional) | 0.05 | 0.3 | — | 0.5 | 0.2 |
| PMN | — | — | 0.08 | — | — |
| Protease A (optional) | — | — | — | — | 0.1 |
| Aldose Oxidase | — | — | 0.3 | — | 0.003 |
| ZnCl2 | 0.1 | 0.05 | 0.05 | 0.05 | 0.02 |
| Ca formate | 0.05 | 0.07 | 0.05 | 0.06 | 0.07 |
| DETBCHD | — | — | 0.02 | 0.01 | — |
| SRP1 | 0.5 | 0.5 | — | 0.3 | 0.3 |
| Boric acid | — | — | — | — | 2.4 |
| Sodium xylene sulfonate | — | — | 3.0 | — | — |
| Sodium cumene | — | — | — | 0.3 | 0.5 |
| sulfonate | | | | | |
| DC 3225C | 1.0 | 1.0 | 1.0 | 1.0 | 1.0 |
| 2-butyl-octanol | 0.03 | 0.04 | 0.04 | 0.03 | 0.03 |
| Brightener 1 | 0.12 | 0.10 | 0.18 | 0.08 | 0.10 |

Balance to 100% perfume/dye and/or water
#1: Add 1N HCl aq. soln to adjust the neat pH of the formula in the range from about 3 to about 5.
The pH of Examples above 6(I)-(II) is about 5 to about 7, and of 6(III)-(V) is about 7.5 to about 8.5.

### Example 7

### Liquid Hand Dishwashing Detergent Compositions Comprising TfuLip2

In this example, various hand dish liquid detergent formulations are provided. In each of these formulations, TfuLip2 is included at a concentration of from about 0.0001 to about 10 weight percent. In some alternative embodiments, other concentrations will find use, as determined by the formulator, based on their needs.

TABLE 7-1
Liquid Hand Dishwashing Detergent Compositions

| | Formulations | | | | | |
|---|---|---|---|---|---|---|
| Compound | I | II | III | IV | V | VI |
| C₁₂-C₁₅ AE_{1.8}S | 30.0 | 28.0 | 25.0 | — | 15.0 | 10.0 |
| LAS | — | — | — | 5.0 | 15.0 | 12.0 |
| Paraffin Sulfonate | — | — | — | 20.0 | — | — |
| C₁₀-C₁₈ Alkyl | 5.0 | 3.0 | 7.0 | — | — | — |
| Dimethyl Amine | | | | | | |
| Oxide | | | | | | |
| Betaine | 3.0 | — | 1.0 | 3.0 | 1.0 | — |
| C₁₂ poly-OH | — | — | — | 3.0 | — | 1.0 |
| fatty acid amide | | | | | | |
| C₁₄ poly-OH | — | 1.5 | — | — | — | — |
| fatty acid amide | | | | | | |
| C₁₁E₉ | 2.0 | — | 4.0 | — | — | 20.0 |
| DTPA | — | — | — | — | 0.2 | — |
| Tri-sodium Citrate | 0.25 | — | — | 0.7 | — | — |
| dehydrate | | | | | | |
| Diamine | 1.0 | 5.0 | 7.0 | 1.0 | 5.0 | 7.0 |
| MgCl₂ | 0.25 | — | — | 1.0 | — | — |
| nprE (optional) | 0.02 | 0.01 | — | 0.01 | — | 0.05 |
| PMN | — | — | 0.03 | — | 0.02 | — |
| Protease A | — | 0.01 | — | — | — | — |
| (optional) | | | | | | |
| Amylase | 0.001 | — | — | 0.002 | — | 0.001 |
| Aldose Oxidase | 0.03 | — | 0.02 | — | 0.05 | — |
| Sodium Cumene | — | — | — | 2.0 | 1.5 | 3.0 |
| Sulphonate | | | | | | |
| PAAC | 0.01 | 0.01 | 0.02 | — | — | — |
| DETBCHD | — | — | — | 0.01 | 0.02 | 0.01 |

Balance to 100% perfume/dye and/or water
The pH of Examples 7(I)-(VI) is about 8 to about 11

### Example 8

### Liquid Automatic Dishwashing Detergent Compositions Comprising TfuLip2

In this example, various liquid automatic dishwashing detergent formulations are provided. In each of these formulations, TfuLip2 polypeptide is included at a concentration of from about 0.0001 to about 10 weight percent. In some alternative embodiments, other concentrations will find use, as determined by the formulator, based on their needs.

TABLE 8-1
Liquid Automatic Dishwashing Detergent Compositions

| | Formulations | | | | |
|---|---|---|---|---|---|
| Compound | I | II | III | IV | V |
| STPP | 16 | 16 | 18 | 16 | 16 |
| Potassium Sulfate | — | 10 | 8 | — | 10 |
| 1,2 propanediol | 6.0 | 0.5 | 2.0 | 6.0 | 0.5 |
| Boric Acid | — | — | — | 4.0 | 3.0 |
| CaCl₂ dihydrate | 0.04 | 0.04 | 0.04 | 0.04 | 0.04 |
| Nonionic | 0.5 | 0.5 | 0.5 | 0.5 | 0.5 |
| nprE (optional) | 0.1 | 0.03 | — | 0.03 | — |
| PMN | — | — | 0.05 | — | 0.06 |
| Protease B (optional) | — | — | — | 0.01 | — |
| Amylase | 0.02 | — | 0.02 | 0.02 | — |
| Aldose Oxidase | — | 0.15 | 0.02 | — | 0.01 |
| Galactose Oxidase | — | — | 0.01 | — | 0.01 |
| PAAC | 0.01 | — | — | 0.01 | — |
| DETBCHD | — | 0.01 | — | — | 0.01 |

Balance to 100% perfume/dye and/or water

### Example 9

### Granular and/or Tablet Laundry Compositions Comprising TfuLip2

This example provides various formulations for granular and/or tablet laundry detergents. In each of these formulations, TfuLip2 is included at a concentration of from about 0.0001 to about 10 weight-percent. In some alternative embodiments, other concentrations will find use, as determined by the formulator, based on their needs.

TABLE 9-1
Granular and/or Tablet Laundry Compositions

| | Formulations | | | | |
|---|---|---|---|---|---|
| Compound | I | II | III | IV | V |
| Base Product | | | | | |
| C₁₄-C₁₅AS or TAS | 8.0 | 5.0 | 3.0 | 3.0 | 3.0 |
| LAS | 8.0 | — | 8.0 | — | 7.0 |
| C₁₂-C₁₅AE₃S | 0.5 | 2.0 | 1.0 | — | — |
| C₁₂-C₁₅E₅ or E₃ | 2.0 | — | 5.0 | 2.0 | 2.0 |
| QAS | — | — | — | 1.0 | 1.0 |
| Zeolite A | 20.0 | 18.0 | 11.0 | — | 10.0 |
| SKS-6 (dry add) | — | — | 9.0 | — | — |
| MA/AA | 2.0 | 2.0 | 2.0 | — | — |
| AA | — | — | — | — | 4.0 |
| 3Na Citrate 2H₂O | — | 2.0 | — | — | — |
| Citric Acid (Anhydrous) | 2.0 | — | 1.5 | 2.0 | — |
| DTPA | 0.2 | 0.2 | — | — | — |
| EDDS | — | — | 0.5 | 0.1 | — |
| HEDP | — | — | 0.2 | 0.1 | — |
| PB1 | 3.0 | 4.8 | — | — | 4.0 |
| Percarbonate | — | — | 3.8 | 5.2 | — |
| NOBS | 1.9 | — | — | — | — |
| NACA OBS | — | — | 2.0 | — | — |
| TAED | 0.5 | 2.0 | 2.0 | 5.0 | 1.00 |
| BB1 | 0.06 | — | 0.34 | — | 0.14 |
| BB2 | — | 0.14 | — | 0.20 | — |
| Anhydrous Na Carbonate | 15.0 | 18.0 | — | 15.0 | 15.0 |
| Sulfate | 5.0 | 12.0 | 5.0 | 17.0 | 3.0 |
| Silicate | — | 1.0 | — | — | 8.0 |
| nprE (optional) | 0.03 | — | 0.1 | 0.06 | — |
| PMN | — | 0.05 | — | — | 0.1 |
| Protease B (optional) | — | 0.01 | — | — | — |
| Protease C (optional) | — | — | — | 0.01 | — |
| Lipase | — | 0.008 | — | — | — |
| Amylase | 0.001 | — | — | — | 0.001 |
| Cellulase | — | 0.0014 | — | — | — |
| Pectin Lyase | 0.001 | 0.001 | 0.001 | 0.001 | 0.001 |
| Aldose Oxidase | 0.03 | — | 0.05 | — | — |
| PAAC | — | 0.01 | — | — | 0.05 |

Balance to 100% Moisture and/or Minors*
*Perfume, dye, brightener/SRP1/Na carboxymethylcellulose/photobleach/MgSO₄/PVPVI/suds suppressor/high molecular PEG/clay.

### Example 10

### Additional Liquid Laundry Detergents Comprising TfuLip2

This example provides further formulations for liquid laundry detergents. In each of these formulations, TfuLip2 is included at a concentration of from about 0.0001 to about 10 weight percent. In some alternative embodiments, other concentrations will find use, as determined by the formulator, based on their needs.

TABLE 10-1
Liquid Laundry Detergents

| | Formulations | | | | | |
|---|---|---|---|---|---|---|
| Compound | I | I | II | III | IV | V |
| LAS | 11.5 | 11.5 | 9.0 | — | 4.0 | — |
| C₁₂-C₁₅AE_{2.85}S | — | — | 3.0 | 18.0 | — | 16.0 |
| C₁₄-C₁₅E_{2.5}S | 11.5 | 11.5 | 3.0 | — | 16.0 | — |
| C₁₂-C₁₃E₉ | — | — | 3.0 | 2.0 | 2.0 | 1.0 |
| C₁₂-C₁₃E₇ | 3.2 | 3.2 | — | — | — | — |
| CFAA | — | — | — | 5.0 | — | 3.0 |
| TPKFA | 2.0 | 2.0 | — | 2.0 | 0.5 | 2.0 |
| Citric Acid | 3.2 | 3.2 | 0.5 | 1.2 | 2.0 | 1.2 |
| (Anhy.) | | | | | | |
| Ca formate | 0.1 | 0.1 | 0.06 | 0.1 | — | — |
| Na formate | 0.5 | 0.5 | 0.06 | 0.1 | 0.05 | 0.05 |
| ZnCl2 | 0.1 | 0.05 | 0.06 | 0.03 | 0.05 | 0.05 |
| Na Culmene | 4.0 | 4.0 | 1.0 | 3.0 | 1.2 | — |
| Sulfonate | | | | | | |
| Borate | 0.6 | 0.6 | 1.5 | — | — | — |
| Na Hydroxide | 6.0 | 6.0 | 2.0 | 3.5 | 4.0 | 3.0 |
| Ethanol | 2.0 | 2.0 | 1.0 | 4.0 | 4.0 | 3.0 |
| 1,2 Propanediol | 3.0 | 3.0 | 2.0 | 8.0 | 8.0 | 5.0 |
| Monoethanol- | 3.0 | 3.0 | 1.5 | 1.0 | 2.5 | 1.0 |
| amine | | | | | | |
| TEPAE | 2.0 | 2.0 | — | 1.0 | 1.0 | 1.0 |
| nprE (optional) | 0.03 | 0.05 | — | 0.03 | — | 0.02 |
| PMN | — | — | 0.01 | — | 0.08 | — |
| Protease A | — | — | 0.01 | — | — | — |
| (optional) | | | | | | |
| Lipase | — | — | — | 0.002 | — | — |
| Amylase | — | — | — | — | 0.002 | — |
| Cellulase | — | — | — | — | — | 0.0001 |
| Pectin Lyase | 0.005 | 0.005 | — | | — | — |
| Aldose Oxidase | 0.05 | — | — | 0.05 | — | 0.02 |
| Galactose | — | 0.04 | | | | |
| oxidase | | | | | | |
| PAAC | 0.03 | 0.03 | 0.02 | — | — | — |
| DETBCHD | — | — | — | 0.02 | 0.01 | — |
| SRP 1 | 0.2 | 0.2 | — | 0.1 | — | — |
| DTPA | — | — | — | 0.3 | — | — |
| PVNO | — | — | — | 0.3 | — | 0.2 |
| Brightener 1 | 0.2 | 0.2 | 0.07 | 0.1 | — | — |
| Silicone | 0.04 | 0.04 | 0.02 | 0.1 | 0.1 | 0.1 |
| antifoam | | | | | | |

Balance to 100% perfume/dye and/or water

### Example 11

### High Density Dishwashing Detergents Comprising TfuLip2

This example provides various formulations for high density dishwashing detergents. In each of these compact formulations, TfuLip2 is included at a concentration of from about 0.0001 to about 10 weight percent. In some alternative embodiments, other concentrations will find use, as determined by the formulator, based on their needs.

TABLE 11-1
High Density Dishwashing Detergents

| | Formulations | | | | | |
|---|---|---|---|---|---|---|
| Compound | I | II | III | IV | V | VI |
| STPP | — | 45.0 | 45.0 | — | — | 40.0 |
| 3Na Citrate | 17.0 | — | — | 50.0 | 40.2 | — |
| 2H₂O | | | | | | |
| Na Carbonate | 17.5 | 14.0 | 20.0 | — | 8.0 | 33.6 |
| Bicarbonate | — | — | — | 26.0 | — | — |
| Silicate | 15.0 | 15.0 | 8.0 | — | 25.0 | 3.6 |
| Metasilicate | 2.5 | 4.5 | 4.5 | — | — | — |
| PB1 | — | — | 4.5 | — | — | — |
| PB4 | — | — | — | 5.0 | — | — |
| Percarbonate | — | — | — | — | — | 4.8 |
| BB1 | — | 0.1 | 0.1 | — | 0.5 | — |
| BB2 | 0.2 | 0.05 | — | 0.1 | — | 0.6 |
| Nonionic | 2.0 | 1.5 | 1.5 | 3.0 | 1.9 | 5.9 |
| HEDP | 1.0 | — | — | — | — | — |
| DETPMP | 0.6 | — | — | — | — | — |
| PAAC | 0.03 | 0.05 | 0.02 | — | — | — |
| Paraffin | 0.5 | 0.4 | 0.4 | 0.6 | — | — |
| nprE (optional) | 0.072 | 0.053 | — | 0.026 | — | 0.01 |
| PMN | — | — | 0.053 | — | 0.059 | — |
| Protease B | — | — | — | — | — | 0.01 |
| (optional) | | | | | | |
| Amylase | 0.012 | — | 0.012 | — | 0.021 | 0.006 |
| Lipase | — | 0.001 | — | 0.005 | — | — |
| Pectin Lyase | 0.001 | 0.001 | 0.001 | — | — | — |
| Aldose Oxidase | 0.05 | 0.05 | 0.03 | 0.01 | 0.02 | 0.01 |
| BTA | 0.3 | 0.2 | 0.2 | 0.3 | 0.3 | 0.3 |
| Poly- | 6.0 | — | — | — | 4.0 | 0.9 |
| carboxylate | | | | | | |
| Perfume | 0.2 | 0.1 | 0.1 | 0.2 | 0.2 | 0.2 |

Balance to 100% Moisture and/or Minors*
*Brightener/dye/SRP1/Na carboxymethylcellulose/photobleach/MgSO₄/PVPVI/suds suppressor/high molecular PEG/clay.
The pH of Examples 11(I) through (VI) is from about 9.6 to about 11.3.

### Example 12

### Tablet Dishwashing Detergent Compositions Comprising TfuLip2

This example provides various tablet dishwashing detergent formulations. The following tablet detergent compositions of the present disclosure are prepared by compression of a granular dishwashing detergent composition at a pressure of 13KN/cm² using a standard 12 head rotary press. In each of these formulations, TfuLip2 is included at a concentration of from about 0.0001 to about 10 weight percent. In some alternative embodiments, other concentrations will find use, as determined by the formulator, based on their needs.

TABLE 12-1
Tablet Dishwashing Detergent Compositions

| | Formulations | | | | | | | |
|---|---|---|---|---|---|---|---|---|
| Compound | I | II | III | IV | V | VI | VII | VIII |
| STPP | — | 48.8 | 44.7 | 38.2 | — | 42.4 | 46.1 | 46.0 |
| 3Na Citrate 2H₂O | 20.0 | — | — | — | 35.9 | — | — | — |
| Na Carbonate | 20.0 | 5.0 | 14.0 | 15.4 | 8.0 | 23.0 | 20.0 | — |
| Silicate | 15.0 | 14.8 | 15.0 | 12.6 | 23.4 | 2.9 | 4.3 | 4.2 |
| Lipase | 0.001 | — | 0.01 | — | 0.02 | — | — | — |
| Protease B (optional) | 0.01 | — | — | — | — | — | — | — |
| Protease C (optional) | — | — | — | — | — | 0.01 | — | — |
| nprE (optional) | 0.01 | 0.08 | — | 0.04 | — | 0.023 | — | 0.05 |
| PMN | — | — | 0.05 | — | 0.052 | — | 0.023 | — |
| Amylase | 0.012 | 0.012 | 0.012 | — | 0.015 | — | 0.017 | 0.002 |
| Pectin Lyase | 0.005 | — | — | 0.002 | — | — | — | — |
| Aldose Oxidase | — | 0.03 | — | 0.02 | 0.02 | — | 0.03 | — |
| PB1 | — | — | 3.8 | — | 7.8 | — | — | 4.5 |
| Percarbonate | 6.0 | — | — | 6.0 | — | 5.0 | — | — |
| BB1 | 0.2 | — | 0.5 | — | 0.3 | 0.2 | — | — |
| BB2 | — | 0.2 | — | 0.5 | — | — | 0.1 | 0.2 |
| Nonionic | 1.5 | 2.0 | 2.0 | 2.2 | 1.0 | 4.2 | 4.0 | 6.5 |
| PAAC | 0.01 | 0.01 | 0.02 | — | — | — | — | — |
| DETBCHD | — | — | — | 0.02 | 0.02 | — | — | — |
| TAED | — | — | — | — | — | 2.1 | — | 1.6 |
| HEDP | 1.0 | — | — | 0.9 | — | 0.4 | 0.2 | — |
| DETPMP | 0.7 | — | — | — | — | — | — | — |
| Paraffin | 0.4 | 0.5 | 0.5 | 0.5 | — | — | 0.5 | — |
| BTA | 0.2 | 0.3 | 0.3 | 0.3 | 0.3 | 0.3 | 0.3 | — |
| Polycarboxylate | 4.0 | — | — | — | 4.9 | 0.6 | 0.8 | — |
| PEG 400-30,000 | — | — | — | — | — | 2.0 | — | 2.0 |
| Glycerol | — | — | — | — | — | 0.4 | — | 0.5 |
| Perfume | — | — | — | 0.05 | 0.2 | 0.2 | 0.2 | 0.2 |

Balance to 100% Moisture and/or Minors*
*Brightener/SRP1/Na carboxymethylcellulose/photobleach/MgSO₄/PVPVI/suds suppressor/high molecular PEG/clay.
The pH of Examples 12(I) through 12(VII) is from about 10 to about 11.5; pH of 12(VIII) is from 8-10.
The tablet weight of Examples 12(I) through 12(VIII) is from about 20 grams to about 30 grams.

### Example 13

### Liquid Hard Surface Cleaning Detergents Comprising TfuLip2

This example provides various formulations for liquid hard surface cleaning detergents. In each of these formulations, TfuLip2 is included at a concentration of from about 0.0001 to about 10 weight percent. In some alternative embodiments, other concentrations will find use, as determined by the formulator, based on their needs.

TABLE 13-1
Liquid Hard Surface Cleaning Detergents

| | Formulations | | | | | | |
|---|---|---|---|---|---|---|---|
| Compound | I | II | III | IV | V | VI | VII |
| C₉-C₁₁E₅ | 2.4 | 1.9 | 2.5 | 2.5 | 2.5 | 2.4 | 2.5 |
| C₁₂-C₁₄E₅ | 3.6 | 2.9 | 2.5 | 2.5 | 2.5 | 3.6 | 2.5 |
| C₇-C₉E₆ | — | — | — | — | 8.0 | — | — |
| C₁₂-C₁₄E₂₁ | 1.0 | 0.8 | 4.0 | 2.0 | 2.0 | 1.0 | 2.0 |
| LAS | — | — | — | 0.8 | 0.8 | — | 0.8 |
| Sodium culmene | 1.5 | 2.6 | — | 1.5 | 1.5 | 1.5 | 1.5 |
| sulfonate | | | | | | | |
| Isachem ® AS | 0.6 | 0.6 | — | — | — | 0.6 | — |
| Na₂CO₃ | 0.6 | 0.13 | 0.6 | 0.1 | 0.2 | 0.6 | 0.2 |
| 3Na Citrate 2H₂O | 0.5 | 0.56 | 0.5 | 0.6 | 0.75 | 0.5 | 0.75 |
| NaOH | 0.3 | 0.33 | 0.3 | 0.3 | 0.5 | 0.3 | 0.5 |
| Fatty Acid | 0.6 | 0.13 | 0.6 | 0.1 | 0.4 | 0.6 | 0.4 |
| 2-butyl octanol | 0.3 | 0.3 | — | 0.3 | 0.3 | 0.3 | 0.3 |
| PEG DME-2000 ® | 0.4 | — | 0.3 | 0.35 | 0.5 | — | — |
| PVP | 0.3 | 0.4 | 0.6 | 0.3 | 0.5 | — | — |
| MME PEG (2000) ® | — | — | — | — | — | 0.5 | 0.5 |
| Jeffamine ® | — | 0.4 | — | — | 0.5 | — | — |
| ED-2001 | | | | | | | |
| PAAC | — | — | — | 0.03 | 0.03 | 0.03 | — |
| DETBCHD | 0.03 | 0.05 | 0.05 | — | — | — | — |
| nprE (optional) | 0.07 | — | 0.08 | 0.03 | — | 0.01 | 0.04 |
| PMN | — | 0.05 | — | — | 0.06 | — | — |
| Protease B | — | — | — | — | — | 0.01 | — |
| (optional) | | | | | | | |
| Amylase | 0.12 | 0.01 | 0.01 | — | 0.02 | — | 0.01 |
| Lipase | — | 0.001 | — | 0.005 | — | 0.005 | — |
| Pectin Lyase | 0.001 | — | 0.001 | — | — | — | 0.002 |
| ZnCl2 | 0.02 | 0.01 | 0.03 | 0.05 | 0.1 | 0.05 | 0.02 |
| Calcium Formate | 0.03 | 0.03 | 0.01 | — | — | — | — |
| PB1 | — | 4.6 | — | 3.8 | — | — | — |
| Aldose Oxidase | 0.05 | — | 0.03 | — | 0.02 | 0.02 | 0.05 |

Balance to 100% perfume/dye and/or water
The pH of Examples 13(I) through (VII) is from about 7.4 to about 9.5.

### Example 14

### Stability of TfuLip2 in Detergents in the Presence and Absence of Protease

The stability of TfuLip2 in detergents was studied in the presence or absence of protease in commercially available detergents, and compared to the stability of a commercial benchmark enzyme LIPEX® (Thermomyces lanuginosus Lip3 lipase; Novozymes, Copenhagen, DK), under similar conditions.

OMO™, Small and Mighty liquid detergent (Unilever) and Ariel color liquid detergent (Procter & Gamble) were heat inactivated prior to use by placing in a water bath at 95° C. for 2 hours. Following heat inactivation, the detergents were tested for protease and lipase activity and found to be negative for all.

TfuLip2 and LIPEX® lipases were added to the detergents at a final concentration of 0.2 ppm. Subtilisin protease (Purafect 4000L; Danisco US. Inc, Genencor Division) was dosed at a final concentration of 1.0 ppm. These concentrations of lipase and protease are typical of those found in detergent wash media, and reflect the real-world operating conditions for enzymes under wash conditions. Detergent mixtures to which lipase or lipase/protease were added were placed at 37° C. for 28 days. Samples were withdrawn at days 0, 2, 7, and 15 and assayed for lipase activity using Tributyrin (CAS 60-01-5) as substrate. The method is based on the speed at which the enzyme hydrolyzes tributyrin. The butyric acid formed by the action of the lipase is titrated with sodium hydroxide and the consumption of NaOH is recorded as a function of time.

An emulsion containing 5% Tributyrin (v/v) in 0.05 M NaCl, 0.5 mM KH₂PO₄, 0.1% Gum arabic, and 9% glycerol was prepared by high sheer mixing of the sample for 20 seconds using a T25 Ultra TURRAX® disperser (IKA®, Germany). 2 mL of enzyme in detergent sample was added to 25 mL of the homogenized substrate and the samples incubated at 30° C. for 6 minutes. The amount of 0.05 M NaOH required to keep the pH of the reaction mixture at 8.0 was determined and enzyme activity was calculated based on the consumption of the NaOH base. The data shown in Table 14-1 represents the percentage remaining lipase activity compared to the activity at day 0 with no protease added (for the respective detergents). TfuLip2 lipase clearly demonstrated better stability than LIPEX® lipase, particularly in the presence of protease.

TABLE 14-1
Percent lipase activity remaining in the presence of detergent and protease

| | Omo liquid | | Omo liquid | | Ariel color | | Ariel color liquid | |
|---|---|---|---|---|---|---|---|---|
| | detergent alone | | detergent + protease | | liquid detergent | | detergent + protease | |
| Enzyme | TfuLip2 | Lipex | TfuLip2 | Lipex | TfuLip2 | Lipex | TfuLip2 | Lipex |
| Day 0 | 100 | 100 | 94 | 111 | 100 | 100 | 101 | 102 |
| Day 2 | 103 | 99 | 106 | 72 | 94 | 97 | 95 | 61 |
| Day 7 | 106 | 84 | 106 | 33 | 90 | 80 | 86 | 18 |
| Day 15 | 76 | 56 | 77 | 4 | 59 | 44 | 57 | 7 |

### Example 15

### Cleaning Performance of TfuLip2 at Different Temperatures

Cleaning performance of TfuLip2 on stained fabrics was tested at 15° C., 20° C., 30° C., and 40° C. in a microswatch assay format in commercially available, heat inactivated Ariel color, liquid and Ariel color, powder detergents. The assay was performed as described in Example 5, with the modification that the plates were shaken at 15, 20, 30 and 40° C., respectively, instead of at 37° C. TfuLip2 was dosed in 0.2 or 0.7 U/ml and μmol free fatty acid/min released from Trioleate, pH 8.2 was measured as described in Example 4. The results are shown in Tables 15-1, 15-2, and 15-3.

TABLE 15-1
% Soil removal in the absence of detergent

| TfuLip2 (U/ml) | 15° C. | 20° C. | 30° C. | 40° C. |
|---|---|---|---|---|
| 0 | 2 | 2 | 8 | 4 |
| 0.2 | 17 | 20 | 15 | 15 |
| 0.7 | 19 | 23 | 24 | 28 |

The results in Table 15-1 show that TfuLip2 demonstrates dose-responsive cleaning performance in the absence of detergent at all temperatures ranging from 15° C. to 40° C. The best performance is achieved with the high dose of enzyme at 40° C.

TABLE 15-2
% Soil removal in the presence of 0.6 g/L Ariel liquid detergent

| TfuLip2 (U/ml) | 15° C. | 20° C. | 30° C. | 40° C. |
|---|---|---|---|---|
| 0 | 9 | 3 | 7 | 3 |
| 0.2 | 18 | 19 | 25 | 27 |
| 0.7 | 21 | 24 | 34 | 39 |

The results in Table 15-2 indicate that TfuLip2 demonstrates dose-responsive cleaning performance in 0.6 g/L Ariel Color Liquid detergent at all temperatures ranging from 15° C. to 40° C. The best performance is achieved with the high dose of enzyme at 40° C. At 30° C. and 40° C., the cleaning performance achieved with TfuLip2 in the presence of 0.6 g/L Ariel Color Liquid detergent is substantially better than that with TfuLip2 in the absence of detergent.

TABLE 15-3
% Soil removal in the presence of 0.6 g/L Ariel powder detergent

| TfuLip2 (U/ml) | 15° C. | 20° C. | 30° C. | 40° C. |
|---|---|---|---|---|
| 0 | 10 | 7 | 12 | 15 |
| 0.2 | 23 | 15 | 24 | 29 |
| 0.7 | 23 | 22 | 32 | 37 |

The results in Table 15-3 show that TfuLip2 demonstrates dose-responsive cleaning performance at 20° C. to 40° C. in 0.6 g/L Ariel Color Powder detergent. The best performance is achieved with the high dose of enzyme at 40° C. At 30° C. and 40° C., the cleaning performance achieved with TfuLip2 in the presence of 0.6 g/L Ariel Color Powder detergent is substantially better than that with TfuLip2 in the absence of detergent.

1. A detergent composition, comprising:
a lipase from Thermobifida fusca, and
a surfactant,
wherein the detergent composition is more effective in removing oily stains from a surface to be cleaned than an equivalent detergent composition lacking the lipase.

2. The detergent composition of claim 1, wherein the lipase is TfuLip2 lipase.

3. The detergent composition of claim 1, wherein the lipase comprises an amino acid sequence having at least 90% amino acid sequence identity to SEQ ID NO: 2 or SEQ ID NO: 3.

4. (canceled)

5. The detergent composition of claim 1, wherein the lipase is a recombinant lipase.

6. The detergent composition of claim 5, wherein the lipase is a recombinant lipase expressed in Bacillus.

7. The detergent composition of claim 1, wherein the surfactant is an ionic or a non-ionic surfactant.

8. The detergent composition of claim 1, wherein the surfactant is one or more surfactants selected from the group consisting of an anionic surfactant, a cationic surfactant, a zwitterionic surfactant, and a combination thereof.

9. The detergent composition of claim 1, wherein the surfactant comprises one or more surfactants selected from the group consisting of sodium dodecyl benzene sulfonate, sodium hydrogenated cocoate, sodium laureth sulfate, C12-14 pareth-7, C12-15 pareth-7, sodium C12-15 pareth sulfate, and C14-15 pareth-4.

10. The detergent composition of claim 1, formulated at a pH of from about 8.0 to about 10.0.

11. (canceled)

12. The detergent composition of claim 1, wherein the detergent composition is selected from the group consisting of a laundry detergent, a dishwashing detergent, and a hard-surface cleaning detergent.

13. The detergent composition of claim 1, wherein the form of the composition is selected from the group consisting of a liquid, a powder, a granulated solid, and a tablet.

14. The detergent composition of claim 1, wherein the detergent composition is effective in hydrolyzing a lipid at a temperature of from about 30° C. to about 40° C.

15. The detergent composition of claim 1, wherein the detergent composition is more effective in hydrolyzing C4 to C16 substrates compared to an equivalent detergent composition comprising Pseudomonas pseudoalcaligenes lipase variant M21L (LIPOMAX™) in place of Thermobifida fusca lipase.

16. The detergent composition of claim 1, further comprising a protease.

17. The detergent composition of claim 16, wherein the protease is a subtilisin protease.

18. The detergent composition of claim 16, wherein the stability of the Thermobifida fusca lipase is greater than the stability of Thermomyces lanuginosus Lip3 lipase (LIPEX®) in an equivalent detergent composition comprising Thermomyces lanuginosus Lip3 lipase in place of Thermobifida fusca lipase.

19. The composition of claim 18, wherein stability is measured in a final wash medium.

20. A method for hydrolyzing a lipid present in a soil or stain on a surface, comprising contacting the surface with the detergent composition of claim 1.

21. A method for performing a transesterification reaction, comprising contacting a donor molecule with detergent composition of claim 1.

22. The method of claim 21, wherein the donor molecule comprises a C4-C16 carbon chain.

23. (canceled)
